(19) United States
(12) Patent Application Publication
(10) Pub. No.: US 2023/0030318 A1
(43) Pub. Date: Feb. 2, 2023

(54) MODIFIED PLOIDY LEVELS OF A CANNABIS PLANT

(71) Applicant: Calyxt, Inc., Roseville, MN (US)

(72) Inventors: Benjamin Campbell, Saint Paul, MN (US); Anders Uppgaard, Minneapolis, MN (US)

(73) Assignee: Calyxt, Inc., Roseville, MN (US)

(21) Appl. No.: 17/788,532

(22) PCT Filed: Dec. 23, 2020
(86) PCT No.: PCT/US2020/066866

§ 371 Date: Jun. 23, 2022

Related U.S. Application Data
(60) Provisional application No. 62/953,012, filed on Dec. 23, 2019.
(60) Provisional application No. 63/070,938, filed on Aug. 27, 2020.

(51) Int. Cl. A01H 1/08; A01H 6/28

(52) U.S. Cl. CPC A01H 1/08; A01H 6/28

(57) ABSTRACT

Embodiments of the present disclosure are directed to a method that includes treating a Cannabis plant, plant part, or plant cell with a solution including a chemical agent configured to modify a ploidy level of the Cannabis plant, plant part, or plant cell. The method further includes, in response to the treatment, modifying the ploidy level of the cannabis plant, plant part, or plant cell.

### BACKGROUND

Industrial hemp is a Cannabis plant variety having less than 0.3% tetrahydrocannabinol (THC). Industrial hemp can be cultivated to produce fiber, grain, or non-intoxicating medicinal compounds, such as cannabidiol (CBD) and terpenes. Cannabis is primarily a dioecious species, meaning that male and female flowers are borne on separate plants. Although stress and chemical treatment can induce pollen-producing flowers on female plants, the production of pollen-producing flowers is an exception to the normally dioecious nature of the plant. Male Cannabis plants flower for a period of two to four weeks, and a single male flower can produce 350,000 pollen grains. Pollen can be carried to female plants by the wind and can travel great distances under favorable conditions. Cannabinoids, including CBD, are concentrated in the female flower tissue. The fertilization of female hemp flowers can result in the creation of seeds and a drop in CBD production by the bud. As such, industrial hemp producers growing hemp to obtain medicinal compounds, such as CBD, generally want to prevent the fertilization of the hemp bud flowers.

In many instances, industrial hemp growers hire workers to walk the field to identify and remove male hemp plants. The labor required for rogueing out male plants can be expensive, and large parts of the field can be lost if a single male hemp plant is left to pollinate the surrounding female plants. Moreover, wild hemp pollen or pollen from neighboring fields can cause accidental pollination. Although industry experts recommend a minimum distance of ten miles between outdoor Cannabis fields, this is not a technically feasible strategy.

Accordingly, there is a need for preventing pollination of female hemp plants that can reduce the labor and logistical costs growers spend to remove male hemp plants from the fields.

### SUMMARY

The present disclosure is directed to overcoming the above-mentioned challenges and needs related to Cannabis plants. This summary is provided to introduce a selection of concepts in a simplified form that are further described below in the Detailed Description.

In some embodiments, a method of modifying ploidy levels of a Cannabis plant comprises treating a Cannabis plant, plant part, or plant cell with a solution including a chemical agent configured to modify a ploidy level of the Cannabis plant, plant part, or plant cell. The method further comprises in response to the treatment, modifying the ploidy level of the Cannabis plant, plant part, or plant cell.

In some embodiments, treating the Cannabis plant, plant part, or plant cell with the solution includes applying the solution to at least a portion of the Cannabis plant, the plant part, or the plant cell, wherein the solution includes the chemical agent, a surfactant, and a polar aprotic solvent.

In some embodiments, the surfactant is a foaming agent, and treating the cannabis plant, plant part, or plant cell with the solution includes applying the solution at least partially in a foam form to the Cannabis plant, plant part, or plant cell.

In some embodiments, the surfactant is Tween 20, and the polar aprotic solvent is Dimethylsulphoxide (DMSO). In some embodiments, the chemical agent is selected from colchicine, acenaphtene, trifluralin, aminoprophosmethyl, pronamide, oryzalin, and nitrous oxide.

The method can further include selecting the Cannabis plant, plant part, or plant cell from a plurality of Cannabis plants, plant parts, or plant cells treated with the solution by identifying the modified ploidy level of the Cannabis plant, plant part, or plant cell. The ploidy level can include 2m or 2p+1 complete chromosome sets, incomplete chromosome sets, or chromosome sets with an additional copy of one or more chromosomes in the chromosome sets. In some embodiments, when the modified ploidy level is 2m, m is at least 2.

In some embodiments, when the modified ploidy level is 2p+1, p is an integer from 1 to 4. In some embodiments, p is 1. In some embodiments, p is 2. In some embodiments, when the modified ploidy level is 2m, m is an integer from 2 to 4. In some embodiments, m is 2. In some embodiments, m is 4.

In some embodiments, the modified ploidy level of the Cannabis plant, plant part, or plant cell includes a tetraploid level, and the method further includes cross breeding a diploid Cannabis plant and the Cannabis plant having the modified ploidy level (e.g., tetraploid). In some embodiments, the method can further include identifying, from the cross breed of the diploid Cannabis plant and the Cannabis plant, at least one offspring Cannabis plant that is triploid and female.

Various embodiments are directed to a method comprising treating a plurality of Cannabis plants, plant parts, or plant cells with a solution including a chemical agent configured to modify a ploidy level of the Cannabis plants, plant parts, or plant cells. The method further comprises selecting at least one of the plurality of Cannabis plants, plant parts, or plant cells that exhibits the modified ploidy level, the modified ploidy level including 2m or 2p+1 complete chromosome sets, incomplete chromosome sets, or chromosome sets with an additional copy of one or more chromosomes in the chromosome sets. Further, when the modified ploidy level is 2m, m is at least 2.

In some embodiments, the modified ploidy level includes a diploid level or a triploid level.

The method can further include cross breeding a diploid Cannabis plant and the Cannabis plant, or a modified Cannabis plant generated from the plant part or plant cell that exhibits the modified ploidy level, to generate an offspring Cannabis plant.

In some embodiments, the method further includes selecting the offspring Cannabis plant, the selected offspring Cannabis plant including a triploid Cannabis plant.

In some embodiments, the selected offspring Cannabis plant is female and sterile.

The method can further include generating clones of the selected offspring Cannabis plant. In some embodiments, the selected at least one of the plurality of Cannabis plants, plant parts, or plant cells is a triploid Cannabis plant.

In some embodiments, treating the plurality of Cannabis plants, plant parts, or plant cells with the solution includes applying the solution at least partially in a foam form to the plurality of Cannabis plants, plant parts, or plant cells, wherein the solution includes the chemical agent, a surfactant and, a polar aprotic solvent.

Other example embodiments are directed to a Cannabis plant, Cannabis plant part, or Cannabis plant cell having 2p+1 complete chromosome sets, incomplete chromosome sets, or chromosome sets with an additional copy of one or more chromosomes in the chromosome sets, such as a Cannabis plant, Cannabis plant part, or Cannabis plant cell generated according to any of the above methods. In some embodiments, p is an integer from 1 to 4. In some embodiments, p is 1.

In some embodiments, the Cannabis plant is a Cannabis sativa plant, a Cannabis indica plant, or a Cannabis ruderalis plant.

In some embodiments, the Cannabis plant is a female Cannabis plant incapable of producing seeds upon fertilization.

In some embodiments, the plant has an increased content of cannabinoids compared to a parent diploid plant grown in the same conditions.

In some embodiments, the Cannabis plant part is a seed or a cutting.

### BRIEF DESCRIPTION OF THE DRAWINGS

Various example embodiments can be more completely understood in consideration of the following detailed description in connection with the accompanying drawings, in which:

FIGS. 1A-1B are flow diagrams illustrating example methods for modifying a ploidy level of a Cannabis plant, plant part, or plant cell, consistent with the present disclosure.

FIG. 2 is a flow diagram illustrating an example method for treating a plurality of Cannabis plants, plant parts, or plant cells, consistent with the present disclosure.

FIGS. 3A-3B are diagrams illustrating example schemes of pollination of a modified Cannabis plant and a wild type Cannabis plant, consistent with the present disclosure.

FIGS. 4A-4B are diagrams illustrating example schemes for generation of a triploid Cannabis plant, consistent with the present disclosure.

FIGS. 5A-7I are graphs illustrating resulting ploidy levels from Group 7A of Table 1, consistent with the present disclosure.

FIGS. 8A-12I are graphs illustrating resulting ploidy levels from Group 7B of Table 1, consistent with the present disclosure.

FIGS. 13A-17I are graphs illustrating resulting ploidy levels from Group 8 of Table 1, consistent with the present disclosure.

FIGS. 18A-20E are graphs illustrating resulting ploidy levels from Cannabis plants treated with a solution of colchicine, Tween 20, and DMSO as compared to resulting ploidy levels from control Cannabis plants, consistent with the present disclosure.

FIG. 21 illustrates an example of cross breeding Cannabis plants and a resulting offspring Cannabis plant part, consistent with the present disclosure.

FIGS. 22A-22I illustrate example offspring from cross breeding Cannabis plants, consistent with the present disclosure.

### DETAILED DESCRIPTION

Aspects of the present disclosure are directed to a variety of methods and resulting Cannabis plants, plant parts, and/or plant cells involving modification of a ploidy level of the Cannabis plant, plant part, and/or plant cell. These methods include treating a Cannabis plant, plant part, or plant cell with a chemical solution that modifies the ploidy level. In some embodiments, the modified ploidy level includes 2m or 2p+1 complete chromosome sets, incomplete chromosome sets, or chromosome sets with an additional copy of one or more chromosomes in the chromosome sets. While the present invention is not necessarily limited to such applications, various aspects of the invention may be appreciated through a discussion of various embodiments using this context.

Accordingly, in the following description various specific details are set forth to describe specific embodiments presented herein. It should be apparent to one skilled in the art, however, that one or more other examples and/or variations of these embodiments can be practiced without all the specific details given below. In other instances, well known features have not been described in detail so as not to obscure the description of the embodiments herein. For ease of illustration, the same reference numerals can be used in different diagrams to refer to the same elements or additional instances of the same element.

Cannabis plants are used for a variety of purposes. Cannabis is a fast growing plant, and can be used as a low cost source of food, building or clothing material, biomass, paint, paper, and other material source, as well as for medicinal or recreational purposes. Modification to the Cannabis plant, plant part, or plant cell can be used to provide particular characteristics. Example characteristics can include cannabinoid content, such as cannabidiol (CBD) or tetrahydrocannabinol (THC) content, height, biomass production, resistance to lodging, faster growth rate, terpene content, and resistance to adverse weather, nutrient stress, water stress, temperature stress, pests, and diseases, among other characteristics. Various embodiments of the present disclosure include application of a chemical treatment to a Cannabis plant, plant part, or plant cell that results in a modified ploidy level. The modified ploidy level can provide particular characteristics in a Cannabis plant directly or indirectly through cross breeding. In some embodiments, the modified or cross-bred Cannabis plant can be polyploidy, infertile or sterile, and/or female.

In some embodiments, a resulting Cannabis plant, plant part, or plant cell has 2m or 2p+1 complete chromosome sets, incomplete chromosome sets, or chromosome sets with an additional copy of one or more chromosomes in the chromosome sets per cell. As used herein, “p” and “m” are integers which are used to define the number of chromosome sets in cells of a Cannabis plant (e.g., a ploidy level). In some embodiments, 2m can include even numbered polyploid levels, and 2p+1 can include odd numbered polyploid levels, For example, when the ploidy level is 2p+1, the plant, plant part, or plant cell can have an odd number of complete or partial chromosome sets per cell (e.g., triploidy). When the ploidy level is 2m, the plant, plant part, or plant cell can have an even number of complete or partial chromosome sets per cell (e.g., tetraploidy). In some embodiments, p is an integer from 1 to 4. In some embodiments, m is an integer from 2 to 4. However, embodiments are not so limited, and p and/or m can be an integer greater than 4.

As used herein, a “ploidy level” refers to and/or includes a number of sets of chromosomes and/or chromosome copies of one or more chromosomes of the chromosome sets in a cell or cells of an organism. A “modified ploidy level” refers to and/or includes a change in the number of sets of chromosomes and/or chromosome copies of one or more chromosomes of the chromosome sets, which can result from the chemical treatment. In some embodiments, the treated Cannabis plants are polyploid Cannabis plants. In some embodiments, the treated Cannabis plants are aneuploid Cannabis plants. As used herein, “polyploid levels” or “polyploidy” refers to and/or includes a condition in which cells of an organism (e.g., the Cannabis plant) have more than two sets of chromosomes, such as triploid, tetraploid, or more. “Aneuploid levels” or “aneuploidy”, as used herein, refers to and/or includes a condition in which cells of an organism (e.g., the Cannabis plant) have chromosome sets with an abnormal copy number of chromosomes in the chromosome set, such as a missing copy or an additional copy of one or more chromosomes in the chromosome sets (e.g., 2n−1, 2n+1, etc.). In some embodiments, the treated Cannabis plants are both polyploid and aneuploid Cannabis plants, such as plants or plant parts of the plants having less than the full 2m or 2p+1 chromosome sets per cell.

As further described herein, the resulting plants (e.g., directly treated or offspring) disclosed herein can be sterile, meaning that the egg or ovule donor plants are incapable of producing seeds. For example, the 2p+1 polyploid Cannabis plants, such as triploid plants, can be sterile. The cause of sterility in triploids occurs as a result of improper chromosome pairing and segregation during meiosis, e.g., during the creation of the gametes (egg/ovule and sperm). During meosis, chromosomes first pair up before the cell divides. In (2n) diploid or (4n) tetraploid organisms, each chromosome has a chromosome pair and division happens evenly. As may be appreciated, n in (2n) refers to the haploid chromosome number. In triploid organisms (3n), such even chromosome pairing is not possible, and the resulting uneven chromosome segregation leads to gametes with unequal numbers of chromosomes. The unequal numbers of chromosomes causes pertubations to gene expression levels, which in turn results in non-viable gametes, reducing or eliminating the chance that a triploid female plant produces seeds.

Throughout the disclosure, the terms “hemp,” “Cannabis,” and “cannabis” are used interchangeably. Wild-type Cannabis is a diploid plant that has a haploid chromosome number of n=10 with a somatic chromosome number of 2n=20. Sexual determination in Cannabis is determined by the XX and XY chromosome system, with female plants being XX and male plants being XY. As used herein, the term “female Cannabis plant” refers to and/or includes a Cannabis plant having an XX genotype, and the term “male Cannabis plant” refers to and/or includes a Cannabis plant having an XY genotype. In some embodiments, in a polyploid Cannabis plant, the ratio of X to Y affects if a plant will be female, “female intersex”, or male. For example, a tetraploid Cannabis plant designated as YYYY is male, XXXX is female, XYYY is male-hermaphroditic, and XXXY is female-hermpahroditic.

Female plants capable of producing seeds are referred to herein as an “ovule donor” (sometimes interchangibly referred to as “egg donor”) or “seed producer”. The terms “pollen donor” and “pollen producer” are used to indicate that the plant is capable of producing pollen. Cannabis pollen producers can be male, e.g., having an XY genotype, or alternatively, pollen production can be induced in a female Cannabis plant (such as XX or XXXX plant) under certain conditions, such as by exposure to chemical or environmental stress, thus resulting in pollen that can be used to produce feminized Cannabis seeds. As used herein, “feminized seeds” are seeds that produce plants that are exclusively female.

As used herein when discussing plants, the term “ovule” refers to and/or includes the female gametophyte, whereas the term “pollen” means the male gametophyte. As used herein, the term “plant part” refers to and/or includes any part of a plant. Examples of plant parts include, but are not limited to a leaf, stem, root, tuber, seed, branch, cutting, pubescence, nodule, leaf axil, flower, pollen, stamen, pistil, petal, peduncle, stalk, stigma, style, bract, carpel, sepal, anther, ovule, rhizome, stolon, shoot, pericarp, endosperm, stamen, and leaf sheath. As used herein, the terms “cross”, “crossing”, or “cross breeding” refer to and/or include the process by which the pollen of one flower on one plant is applied (artificially or naturally) to the ovule (stigma) of a flower on another plant.

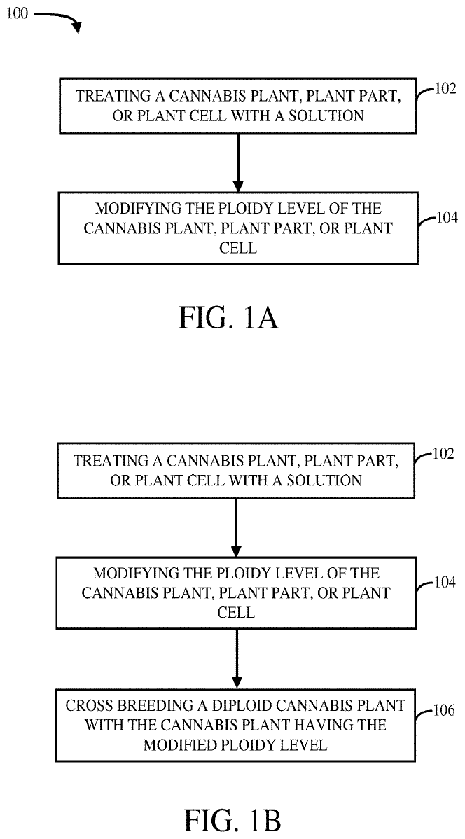

Turning now the figures, FIGS. 1A-1B are flow diagrams illustrating example methods for modifying a ploidy level of a Cannabis plant, plant part, or plant cell, consistent with the present disclosure. In some embodiments, the Cannabis plants disclosed herein are Cannabis sativa plants, Cannabis indica plants, or Cannabis ruderalis plants.

FIG. 1A illustrates an example method 100 for modifying a ploidy level of a Cannabis plant, plant part, or plant cell.

At 102, the method 100 includes treating a Cannabis plant, plant part, or plant cell with a solution that includes a chemical agent. The chemical agent is configured to modify a ploidy level of the Cannabis plant, plant part, or plant cell. In some embodiments, a plurality of Cannabis plant clones can be grown for a period of time (e.g., two to three weeks) and then treated with the solution. In various embodiments, (2n) diploid Cannabis plants, plant parts, or plant cells are treated with the solution including the chemical agent to produce cannabis plants, plant parts, or plant cells with various ploidy levels. Treatment of the diploid cannabis plants, plant parts, or plant cells with the chemical agent can disrupt the meiosis process. Example chemical agents include, but are not limited to, colchicine, acenaphtene, trifluralin, aminoprophosmethyl, pronamide, oryzalin, and nitrous oxide.

Embodiments are not limited to chemical treatment of a diploid Cannabis plant. In various embodiments, the method 100 can include chemical treatment of one or a plurality of polyploid (e.g., tetraploid) Cannabis plants, plant parts and/or plant cells, which results in Cannabis plants, plant parts, or plant cells with the various ploidy levels. As an example, a chemical treatment of plant cells can produce cells (and subsequent plants) with various ploidy levels, from which plants with 2m or 2p+1 ploidy levels can be selected.

Treating the Cannabis plant, plant part, or plant cell with the solution can include applying the solution to at least a portion of the Cannabis plant, the plant part, or the plant cell. In some embodiments, the solution includes the chemical agent, a surfactant, and a polar aprotic solvent. As described above, the chemical agent can disrupt the meiosis process, resulting in different ploidy levels (e.g., different polyploid levels and/or aneuploid levels). The surfactant can break surface tension and act as an emulsifier and, optionally, a foaming agent. The polar aprotic solvent can impact cell permeability by weakening cell membranes. In some embodiments, the surfactant is Tween 20, and the polar aprotic solvent is DMSO. In various embodiments, the surfactant is a foaming agent, and treating the Cannabis plant, plant part, or plant cell with the solution includes applying the solution at least partially in a foam form to the Cannabis plant, plant part, or plant cell.

In some embodiments, the solution includes 0.1-0.5 percent weight per volume (% w/v) colchicine, 0.1-5% w/v Tween 20, and 0.1-5% w/v DMSO. The addition of Tween 20 and DMSO to the solution applied to the Cannabis plants can increase the rate of polyploidy changes by enhancing emulsification and impacting the cell permeability within the Cannabis plant, plant part, or plant cell. While not essential to achieve polyploidy changes, the solution can be pipetted (or repeat pipetted) on Cannabis plants or plant parts, such that the treatment point (e.g., the meristem or seed) is covered in a foam form of the solution. A foam form can have a better adherence to the Cannabis plant than a liquid form of the solution. Tween 20 can act as a foaming agent and allow for generating the foam form of the solution. In some embodiments, the solution can be pipetted on the plant or plant part multiple times a day for multiple consecutive days. For example, 50-100 microliters (μl) of the solution can be pipetted, at least partially in the foam form (e.g., in a foam form, or in a part foam form and a part liquid form), on meristems of the Cannabis plant in the morning and in the evening (e.g., approximately 12 hours apart) and/or for multiple days (e.g., two or three consecutive days).

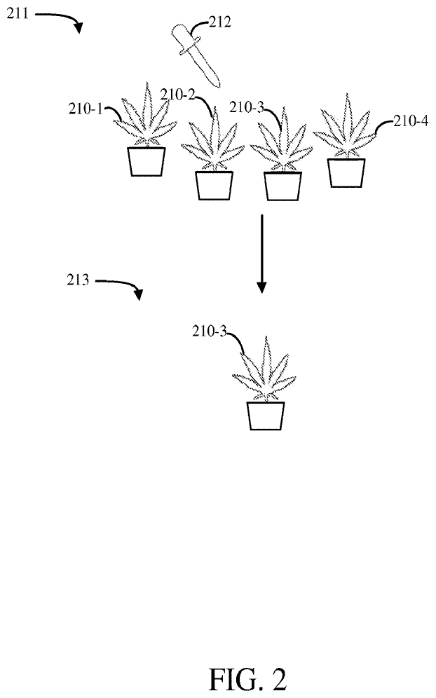

At 104, in response to the treatment, the method 100 includes modifying the ploidy level of the Cannabis plant, plant part, or plant cell. In some embodiments, the chemical treatment can result in different polyploid levels and/or different aneuploid levels. In some embodiments, as further illustrated by FIG. 2, a plurality of Cannabis plants, plant parts, or plant cells are treated with the solution. In such embodiments, the method 100 can further include selecting the Cannabis plant, plant part, or plant cell from the plurality treated with the solution by identifying the modified ploidy level of the Cannabis plant, plant part, or plant cell includes 2m or 2p+1 complete chromosome sets, incomplete chromosome sets, or chromosome sets with an additional copy of one or more chromosomes in the chromosome sets. In some embodiments, p is an integer from 1 to 4. In some embodiments, p is 1. In some embodiments, p is 2. In some embodiments, m is at least 2. In some embodiments, m is an integer from 2 to 4. In some embodiments, m is 2. In some embodiments, m is 4.

In various embodiments, the plurality of Cannabis plants or plant parts are treated with the solution, and allowed to grow and recover for a period of time, such as a number of weeks. Plants that survive the treatment and generate healthy shoots can be selected and screened for modified ploidy levels. In some embodiments, the screening can include collecting leaf tissue, and testing the leaf tissue to identify the ploidy level using flow cytometry. In some embodiments, flow cytometry can be performed by combining the leaf tissue with nuclei extraction buffer and staining buffer and imaging the sample using a flow cytometer that counts the cell nuclei and measures fluorescent strength to indicate ploidy levels, such as using at least some of the features and elements as described by Galbraith D W and Lambert G M (2012), “Using the BD Acurri™ C6 Cytometer for Rapid and Accurate Analysis of the Nuclear DNA Contents of Flowering Plants”, BD Biosciences, which is hereby incorporated by reference in its entirety for its teaching. However, embodiments are not limited to performing flow cytometry, and the resulting Cannabis plant, plant part, or plant cell can be selected based on other techniques, such as phenotyping.

In some embodiments, the resulting Cannabis plants, plant parts, or plant cells from the chemical treatment can include a mixture of the different ploidy levels and/or with complete chromosome sets, incomplete chromosome sets, and/or additional chromosome copies of one or more chromosomes of the chromosome sets (e.g., different polyploid levels and/or different aneuploid levels). Polyploid Cannabis plants can thereby include the complete chromosome sets, incomplete chromosome sets and/or chromosome sets with additional chromosome copies. For example, for a 3n plant with n=10, a resulting triploid Cannabis plant can include 30 chromosomes for the complete chromosome set. In other embodiments or in addition, the resulting triploid Cannabis plant can include a triploid aneuploidy plant, such as a Cannabis plant having an incomplete chromosome set of 29 chromosomes or 28 chromosomes or additional copies of chromosomes, such as 31 chromosomes or 32 chromosomes.

Embodiments in accordance with the present disclosure are directed to induction of tetraploidy and other polyploidies in a plant using various techniques. As described by method 100 of FIG. 1A, antimitotic chemical agents can be used to induce polyploidization of Cannabis. Polyploidy can be induced through application of the antimitotic chemical agent to seeds, seedlings, in vivo shoot tips, or in vitro explants. Somewhat surprisingly, in some embodiments, the treatment of the Cannabis plant parts or plant cells with the chemical agent can result in a plurality of different ploidy levels, including 2m and/or 2p+1 polyploidization. In such embodiments, 2p+1 polyploidization of Cannabis plants can be achieved without crossing a diploid Cannabis plant with a polyploid Cannabis plant. However, embodiments are not so limited. For example and as further described herein, the method 100 can further include cross breeding a diploid Cannabis plant with the Cannabis plant having the modified ploidy level.

Embodiments are not limited to the above described chemical treatment. In some embodiments, the chemical treatment can include different methodologies, chemical agents, and/or treatment solutions, among other variations from that described by method 100. For example, various embodiments are directed to a chemical application or treatment using different types of chemical agents and/or solutions. In some embodiments, tetraploidization can be induced by treatment with oryzalin, for example, as described by Parsons J L., Martin S L., James T., Golenia G., Boudko E A., Hepworth S R, “Polyploidization for the Genetic Improvement of Cannabis sativa”, Frontiers in Plant Science, 2019, 10: 476, which is hereby incorporated by reference in its entirety for its teaching.

In some embodiments, Cannabis plants, plant parts, or plant cells can be treated with nitrous oxide. The nitrous oxide can be in a gaseous form (e.g., under pressure). For example, the Cannabis plant, plant part, or plant cell can be sealed in a chamber with nitrous oxide and oxygen for a period of time and while the chamber is pressurized. In some embodiments, the Cannabis plants, plant parts, or plant cells are treated with nitrous oxide at 2-10 atmospheric pressure (atms) for between 2 to 78 hours. In some embodiments, the plants or plant parts can be treated a few hours to days after pollination. In some embodiments, the plant or plant parts can be treated prior to pollination and/or while in a vegetative state. In some embodiments, the plants can be treated as described by Okazaki K, Nukui S, Ootuka H (2012), “Application of nitrous oxide gas as a polyploidizing agent in tulip and lily breeding”, Floric Ornam Biotechnol, 6:39-43, which is hereby incorporated by reference in its entirety for its teaching, although embodiments are not so limited.

In various embodiments, different types, amounts, or ranges of the chemical agent and/or applications per day can be used. As an example, for acenaphtene, trifluralin, aminoprophosmethyl, pronamide, and oryzalin, the amount of chemical agent in the solution can be in a range that is less than 0.1-0.5% w/v, such as a magnitude less (e.g., 0.001-0.05% w/v). In some embodiments, the range of chemical agent (e.g., colchicine) in the solution can include 0.15-0.5% w/v, 0.2-0.5% w/v, 0.25-0.5% w/v, 0.3-0.5% w/v, 0.4-0.5% w/v, 0.1-0.45% w/v, 0.1-0.4% w/v, 0.1-0.3% w/v, 0.1-0.25% w/v, 0.15-0.45% w/v, 0.2-0.4% w/v, 0.25-0.3% w/v, among other variations within the range of 0.1-0.5% w/v.

In some embodiments, different amounts or ranges of the surfactant and/or the polar aprotic solvent can be used. In some embodiments, the range of the surfactant (e.g., Tween 20) in the solution can include 0.25-5% w/v, 0.5-5% w/v, 0.75-5% w/v, 1-5% w/v, 2-5% w/v, 3-5% w/v, 0.1-4.5% w/v, 0.1-4% w/v, 0.1-3.5% w/v, 0.1-3% w/v, 0.1-2.5% w/v, 0.1-2% w/v, 0.1-1.5% w/v, 0.1-1% w/v, 0.25-4% w/v, 0.5-3% w/v, 1-3% w/v, 1-2% w/v, among other variations within the range of 0.1-5% w/v. In some embodiments, the range of polar aprotic solvent (e.g., DMSO) in the solution can include 0.25-5% w/v, 0.5-5% w/v, 0.75-5% w/v, 1-5% w/v, 2-5% w/v, 3-5% w/v, 0.1-4.5% w/v, 0.1-4% w/v, 0.1-3.5% w/v, 0.1-3% w/v, 0.1-2.5% w/v, 0.1-2% w/v, 0.1-1.5% w/v, 0.1-1% w/v, 0.25-4% w/v, 0.5-3% w/v, 1-3% w/v, 1-2% w/v, among other variations within the range of 0.1-5% w/v.

In some embodiments, the chemical agent includes 0.1-0.5% w/v colchicine, 0.1-0.25% w/v colchicine, or 0.25% w/v colchicine. In some embodiments, the surfactant includes 0.1-3% w/v Tween 20, 1-2% w/v Tween 20, or 1% w/v Tween 20. In some embodiments, the polar aprotic solvent includes 0.1-2% w/v DMSO, 1-2% w/v DMSO, 2% w/v DMSO, or 1% w/v DMSO. However, embodiments are not so limited and can include various ranges. The above ranges are provided as non-limiting examples.

FIG. 1B illustrates an example variation of the method 100 of FIG. 1A for modifying a ploidy level of a Cannabis plant, plant part, or plant cell. As with FIG. 1A, the method includes treating the Cannabis plant, plant part, or plant cell with the solution, at 102, and modifying the ploidy level, at 104.

At 106, the method further includes cross breeding a diploid Cannabis plant and the Cannabis plant having the modified ploidy level. For example, the modified ploidy level of the Cannabis plant, plant part, or plant cell can include 2m, such as a tetraploid level. In such embodiments, the method 100 can further include identifying at least one offspring Cannabis plant that is 2p+1 from the cross breed of the diploid Cannabis plant and the Cannabis plant. For example, the identified offspring Cannabis plant can be triploid and female.

As used herein, the term “offspring” or “offspring Cannabis plant” refers to and/or includes a plant resulting as progeny from a vegetative or sexual reproduction from one or more parent plants or descendants thereof. For instance, an offspring plant can be obtained by crossing two parent plants, and includes selfings as well as the first generation (F1), second generation (F2), or still further generations. An F1 is a first-generation offspring produced from parent plants, at least one of which has the modified ploidy level. Offspring of second generation (F2) or subsequent generations (F3, F4, etc.) are specimens produced from selfings of F1s, F2s, etc. An F1 can be a 2p+1 offspring resulting from a cross between a 2m polyploid parent plant and a (2n) diploid parent plant, with the F1 being sterile.

In some embodiments, a triploid plant can be generated by crossing a diploid parent plant and a tetraploid parent plant that is generated using the above described methods. Each parent plant contributes half of its deoxyribonucleic acid (DNA) to its gametes, with a tetraploid plant generating 2n gametes and the diploid plant generating 1n gametes. When the 2n gamete fuses with the 1n gamete, a 3n zygote is generated. The pollen donor can be either the 2n or the 4n plant, as further described below. The 3n zygote can be used to generate a 3n Cannabis plant and/or clones. However, embodiments are not so limited and can include crossing of other 2m Cannabis plants and generating other 2p+1 offspring plants. As used herein, a “zygote” refers to and/or includes a cell formed by a fertilization event between two gametes, and which includes a genome that is a combination of the DNA in each gamete. The zygote can mature into a sporophyte, and further development leads to a seed.

In some embodiments, the method 100 and various variations can be used to generate 2p+1 polyploid Cannabis plants, plant cells, or plant parts, directly through the application of the solution and selection of a 2p+1 Cannabis plant, plant part, or plant cell. In some embodiments, as shown by FIG. 1B, a method and various variations can be used to generate 2p+1 polyploid Cannabis plants, plant cells, or plant parts indirectly through the application of the solution and selection of a 2m Cannabis plant, plant part, or plant cell, followed by cross breeding a diploid Cannabis plant with the 2m Cannabis plant, wherein m is at least 2. In some embodiments, p and m can include different integers. In some embodiments, p and m can be the same integer.

In some embodiments, the 2m or 2p+1 polyploid Cannabis plant parts of the disclosure can be used to generate 2p+1 polyploid Cannabis plants, plant cells, and plant parts. For example, 2p+1 polyploid Cannabis plants can be grown from a seed. In some embodiments, the polyploid Cannabis plants disclosed herein can be produced or propagated by cloning. For example, a 2m or 2p+1 polyploid Cannabis plant can be generated by treating a Cannabis plant part, such as a seed, with a chemical agent to modify the plant ploidy level. The 2p+1 polyploid Cannabis plant can then be produced or propagated from the treated seed.

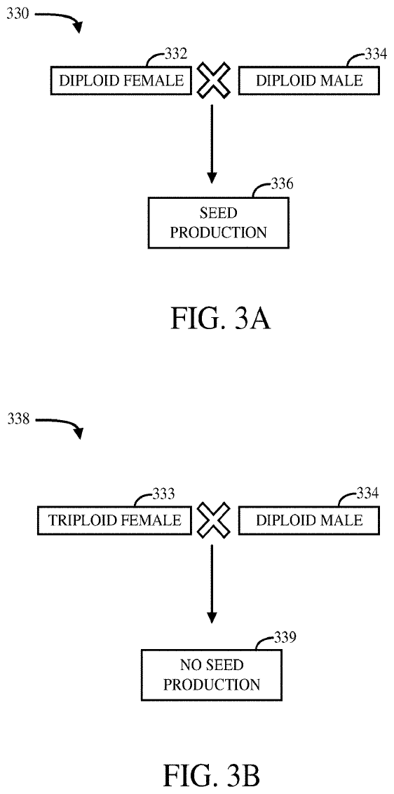

The resulting 2p+1 polyploid Cannabis plants, plant cells, or plant parts can offer certain growing advantages compared to parent or wild diploid plants, as further illustrated by FIGS. 3A-3B. In some embodiments, the Cannabis plants disclosed herein have an increased content of cannabinoids compared to a parent diploid plant grown in the same conditions. In various embodiments, the plants are female plants. In some embodiments, the content of cannabinoids in the female plants of the present disclosure is not affected by contact with Cannabis pollen, as further illustrated by FIG. 3B. For example, the 2p+1 polyploid Cannabis plants, plant parts, or plant cells can be sterile and/or are female plants incapable of producing seeds upon fertilization. In some embodiments, the polyploid Cannabis plants disclosed herein have one or more desired characteristics compared to a parent diploid plant used to generate the plants of the disclosure, such as taller height, higher biomass production, higher resistance to lodging, faster growth rate, higher terpene content, and higher resistance to adverse weather, nutrient stress, water stress, temperature stress, pests, and diseases.

FIG. 2 is a flow diagram illustrating an example method for treating a plurality of Cannabis plants, plant parts, or plant cells, consistent with the present disclosure. In some embodiments, the method of FIG. 2 can include an implementation of the method 100 of FIG. 1A.

At 211, the method includes treating a plurality of Cannabis plants, plant parts, or plant cells with a solution including a chemical agent configured to modify a ploidy level of the Cannabis plants, plant parts, or plant cells, as shown by use of the dropper 212 to apply a solution to the plurality of Cannabis plants 210-1, 210-2, 210-3, 210-4 (herein generally referred to as “the Cannabis plants 210” for ease of reference). As previously described, the solution can include the chemical agent, a surfactant, and polar aprotic solvent and/or which can be applied at least partially in a foam form, although embodiments are not so limited.

After the treatment, at 213, the method includes selecting at least one of the plurality of Cannabis plants, plant parts, or plant cells, e.g., the Cannabis plants 210, that exhibits the modified ploidy level, as shown by the particular Cannabis plant 210-3. The modified ploidy level can include 2m or 2p+1 complete chromosome sets, incomplete chromosome sets, or chromosome sets with an additional copy of one or more chromosomes in the chromosome sets. In some embodiments, the modified ploidy level can include a diploid level or a triploid level.

As described above in connection with FIG. 1A, the treated plants, plant parts, or plant can include a mixture of different ploidy levels. The plurality of treated Cannabis plants 210 can be screened to identify and select tetraploid (or other 2m polyploid) Cannabis plants or triploid (or other 2p+1 polyploid) Cannabis plants. For example, the shoots of the treated Cannabis plants can be tagged for identification purposes, and then tested to identify 2m or 2p+1 polyploid Cannabis plants. In various embodiments, the identified 2m or 2p+1 polyploid Cannabis plants can be selected and cloned. In some embodiments, the polyploid levels of the Cannabis plants, plant parts, or plant cells can be identified via flow cytometry analysis. In some embodiments, the ploidy levels can be identified based on phenotype changes can be used to determine if changes to ploidy level have occurred. Example phenotype changes can include changes to guard cell size, leaf size or shape, seedling size or shape, stomate size or shape, and/or pollen grain size, among other changes. In some embodiments, modifications to the ploidy level can be determined by counting the number of chromosomes present in the plant cells. Various different methodologies, such as fluorescent in situ hybridization (FISH), genomic in situ hybridization (GISH) and/or multi-color FISH (mc-FISH), can be used for determining chromosome counts of resulting plants, plant parts or plant cells, such as from root tips of the resulting or produced plants.

In various embodiments, the method of FIG. 2 can further include cross breeding a diploid Cannabis plant and the Cannabis plant (or a modified Cannabis plant generated from the plant part or plant cell that exhibits the modified ploidy level) to generate an offspring Cannabis plant. In some embodiments, the modified ploidy level includes a tetraploid level, and the tetraploid Cannabis plant is cross bred with a diploid Cannabis plant. The method can further include selecting the offspring Cannabis plant. The offspring Cannabis plant can be selected based on the ploidy level, the sex, among other characteristics or traits. For example, the selected Cannabis plant can be a triploid Cannabis plant, is female, and/or is sterile. In some embodiments, the method can further include generating clones of the selected offspring Cannabis plant.

However embodiments are not limited to performing cross breeding. In some embodiments, the chemical treatment can result in triploid Cannabis plant, plant parts, or plant cells without cross breeding. In such embodiments, the method of FIG. 2 can include selecting the at least one of the plurality of Cannabis plants, plant parts, or plant cells that is a triploid (or other 2p+1) Cannabis plant, plant part, or plant cell at 213 of FIG. 2.

FIGS. 3A-3B are diagrams illustrating example schemes of pollination of a modified Cannabis plant and a wild type Cannabis plant, consistent with the present disclosure. In various embodiments, pollination of the 2p+1 polyploid Cannabis plants of the present disclosure may not result in seed production. For example, as shown by the method 338 of FIG. 3B, a triploid Cannabis female plant 333 (which is generated by methods disclosed herein) is crossed with a diploid wild male plant 334. In response to the cross, no seed production may occur, as shown at 339. By comparison, as shown by the method 330 of FIG. 3A, pollination of a diploid female Cannabis plant 332 with a diploid wild male plant 334 results in seed production. As shown at 336, in response to the cross, seed production may occur.

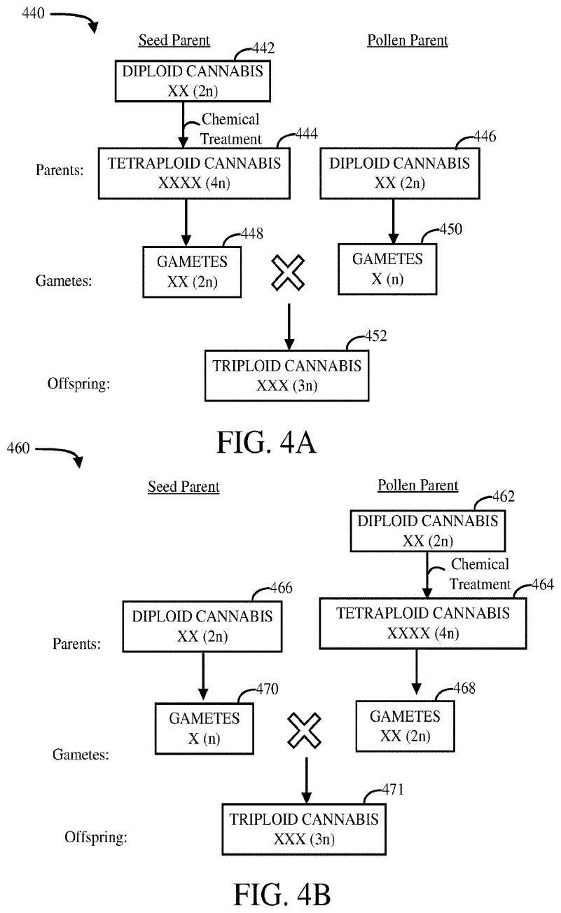

FIGS. 4A-4B are diagrams illustrating example schemes for generation of a triploid Cannabis plant, consistent with the present disclosure. In some embodiments, the 2p+1 Cannabis plant seeds or plants of the present disclosure can be produced by crossing a diploid Cannabis plant and a polyploid Cannabis plant modified by a chemical treatment, e.g., a tetraploid Cannabis plant. Exemplary seeds, such as triploid seeds, can be produced by crossing (2n) diploid lines containing 2n chromosomes per cell with (4n) tetraploid lines containing 4n chromosomes per cell or by crossing (4n) tetraploid lines containing 4n chromosomes per cell with (2n) diploid lines containing 2n chromosomes per cell. In some embodiments, the plant part is a seed, such as a feminized seed which is produced by crossing a female ovule donor and female pollen donor.

In some embodiments, a sterile Cannabis plant, plant part, or plant cell, can be generated by crossing a tetraploid Cannabis plant and a diploid Cannabis plant or plant parts thereof. As shown by FIG. 4A, in some embodiments, the diploid Cannabis plant 446 is used as the pollen donor, and the tetraploid Cannabis plant 444 is used as the ovule donor (e.g., seed parent) to generate the triploid Cannabis seeds 452 that can in turn can be used to generate the triploid Cannabis plants. For example, the method 440 illustrated by FIG. 4A can include treating at least one diploid Cannabis plant 442 with a solution including a chemical agent, as described by the method 100 of FIG. 1, to generate a tetraploid Cannabis plant 444. The tetraploid Cannabis plant 444 is cross bred with the diploid Cannabis plant 446 via a gamete 448 (e.g., ovule) of the tetraploid Cannabis plant 444 and a gamete 450 (e.g., pollen) of the diploid Cannabis plant 446, resulting in triploid Cannabis seed(s) 452.

As shown by FIG. 4B, in some embodiments, the tetraploid Cannabis plant 464 is used as the pollen donor (e.g., the pollen parent), and the diploid Cannabis plant 466 is used as the ovule donor to generate the triploid Cannabis seeds 471 that can in turn can be used to generate the triploid Cannabis plants. For example, the method 460 illustrated by FIG. 4B can include treating at least one diploid Cannabis plant 462 with a solution including a chemical agent, as described by the method 100 of FIG. 1, to generate a tetraploid Cannabis plant 464. The tetraploid Cannabis plant 464 is cross bred with the diploid Cannabis plant 466 via a gamete 470 (e.g., ovule) of the diploid Cannabis plant 466 and a gamete 468 (e.g., pollen) of the tetraploid Cannabis plant 464, resulting in triploid Cannabis seed(s) 471.

For large-scale commercial production of triploid Cannabis seeds 452, 471, tetraploid parental lines 444, 464 and diploid parental lines 446, 466 can be planted in adjacent rows, for example, one or two rows of pollen producers for multiple rows of seed producers, and allowed to cross pollinate.

Although FIGS. 4A-4B illustrate treatment and crossing of a 4n Cannabis plant, embodiments are not so limited and can include treating a plant part or plant cells, and propagating or otherwise generating a Cannabis plant from the modified plant part and/or generating the plant or plant parts from the modified plant cells. Other embodiments can include crossing other types of 2m polyploid (e.g., 8n) Cannabis plants to generate 2p+1 offspring.

In some embodiments, as illustrated by the sex chromosomes (e.g., X, XX, XXX, and XXXX) shown by both FIGS. 4A and 4B, both the ovule donor and the pollen donor are female, and the resulting triploid Cannabis seeds are feminized seeds. A female cannabis plant can be forced into generation of pollen under certain conditions, such as upon contacting with a chemical stressor or under environmental stress (e.g., by treatment with a silver salt such as silver thiosulfate or silver nitrate, rodelization, treatment with colloidal silver, or treatment with gibberellic acid). For example, pollen sac formation in a female Cannabis plant can be induced using the protocols as those described in Mohan Ram H Y, et al., “Induction of fertile male flowers in genetically female Cannabis sativa plants by silver nitrate and silver thiosulphate anionic complex”, Theor Appl Genet., 1982; 62(4): 369-75, which is hereby incorporated herein by reference in its entirety.

The use of the term “or” in the claims and specification is used to mean “and/or” unless explicitly indicated to refer to alternatives only or the alternatives are mutually exclusive, although the disclosure supports a definition that refers to only alternatives and “and/or.”

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Various embodiments are implemented in accordance with the underlying provisional applications, U.S. Provisional Application No. 62/953,012, filed on Dec. 23, 2019, and entitled “Seed-Free Cannabis and Methods”, and U.S. Provisional Application No. 63/070,938, filed on Aug. 27, 2020, and entitled “Polyploid Cannabis and Methods” to each of which benefit is claimed and each are fully incorporated herein by reference in their entireties. For instance, embodiments herein and/or in the provisional applications, including the Appendices A-N can be combined in varying degrees (including wholly). Embodiments discussed in the provisional applications are not intended, in any way, to be limiting to the overall technical disclosure, or to any part of the claimed invention unless specifically noted. As may be appreciated, in some embodiments, the 2p+1 ploidy level described herein can include or be implemented at the 2n+1 ploidy level described in the underlying provisional applications, with integer “p” being replaced by “n”.

While illustrative embodiments have been illustrated and described, it will be appreciated that various changes can be made therein without departing from the scope of the invention.

### EXPERIMENTAL EMBODIMENTS

Various experimental embodiments were conducted that involved applying a chemical treatment to Cannabis plants, plant parts, or plant cells to disrupt meiosis and to provide different ploidy levels. In example embodiments, diploid Cannabis plant clones were grown in flats for two to three weeks. The meristems of the Cannabis plant clones were then treated with 50-100 μl of solution comprising 0.1-0.5% w/v colchicine, 0.1-5% w/v Tween 20, and 0.1-5% w/v DMSO twice a day for three consecutive days. The solution was applied by pipetting a foam form of the solution onto the meristems approximately 12 hours apart (e.g., 6 am and 6 pm). The treated Cannabis plants were then allowed to grow and recover for several weeks. The healthy shoots of the treated Cannabis plants that survived were tagged, for identification purposes, and then screened for changes in ploidy level, e.g., tested to identify 2m or 2p+1 polyploid plants. Shoots selected to be tested were tagged, and fresh leaf tissue was collected. As illustrated further herein, resulting Cannabis plants or plant parts, after the chemical treatment, include a mixture of different ploidy levels.

Flow cytometry was used to identify changes to ploidy level. Flow cytometry is a method to characterize the physical or chemical properties of a population of cells. Samples are prepared using a method similar to that described by Galbraith D W and Lambert G M (2012), “Using the BD Acurri™ C6 Cytometer for Rapid and Accurate Analysis of the Nuclear DNA Contents of Flowering Plants”, BD Biosciences. In experiments, a small amount of leaf tissue was placed into a petri dish. Nuclei extraction buffer was added to the petri dish, and the leaf sample was finely chopped using a razor blade. The sample was incubated for 30 seconds and filtered through a 30 micrometer (μm) filter. A staining buffer was added to the sample and incubated for another 30 seconds. The sample was then analyzed in a flow cytometer. The staining buffer used in the sample preparation process was a fluorescent dye that binds to DNA. The cells were run through a flow cytometer which counts the cell nuclei and measures the fluorescence strength. Fluorescence strength was used as a proxy for DNA content. The fluorescence strength of several hundred or several thousand cell nuclei are measured, creating a distribution of fluorescence strengths with a primary strong peak under a certain fluorescence strength (for non-chimeric plants). A plant with a known ploidy level (2n in the experiments) was run as a control, and the resulting fluorescent distribution and peak position were used as a baseline comparison for all the test samples. Test samples that show a similar primary fluorescence peak position to the 2n control were determined to have a ploidy of 2n. Cells that have twice the DNA content were expected to have twice the fluorescence signature when stained with a fluorescent dye. Thus, leaves that produce cell nuclei with a fluorescence peak strength twice that of the 2n leaf is indicative of the leaf being 4n.

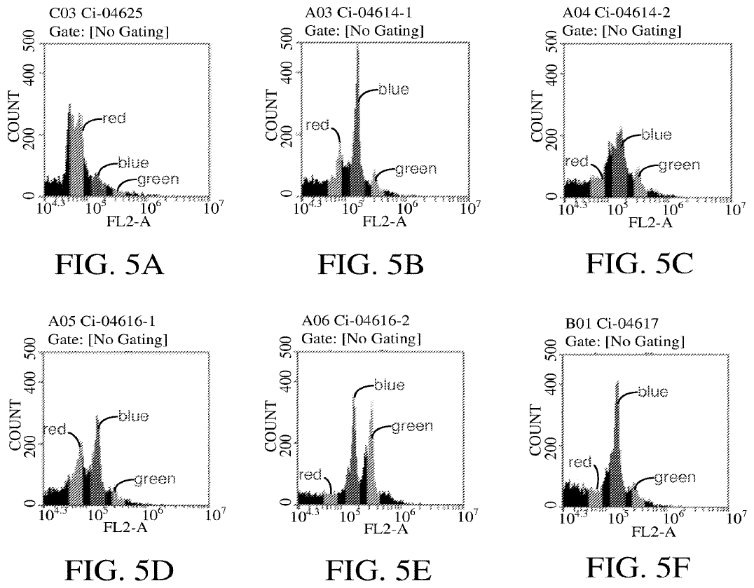

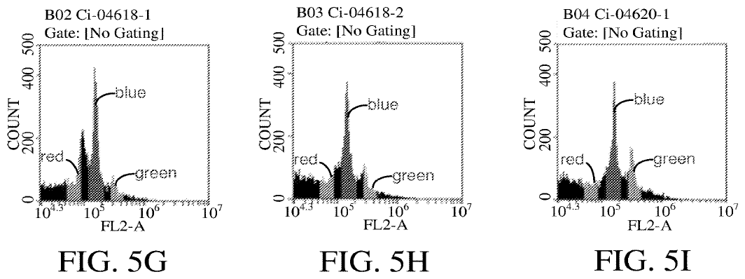

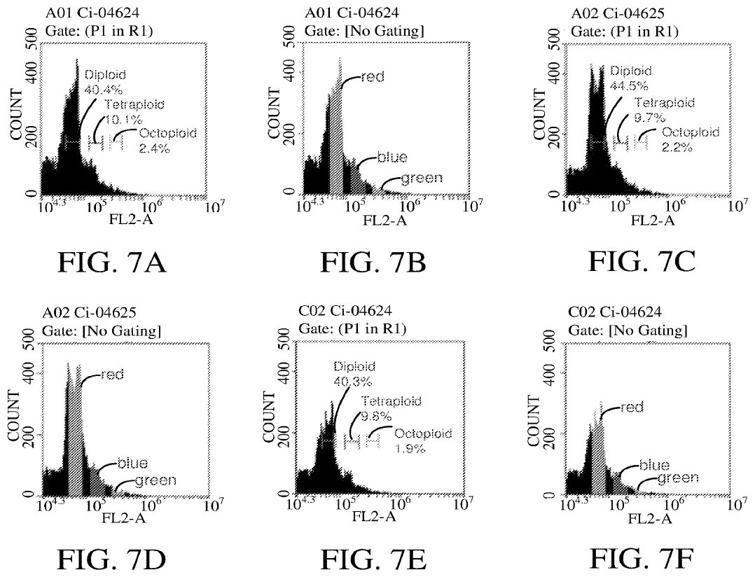

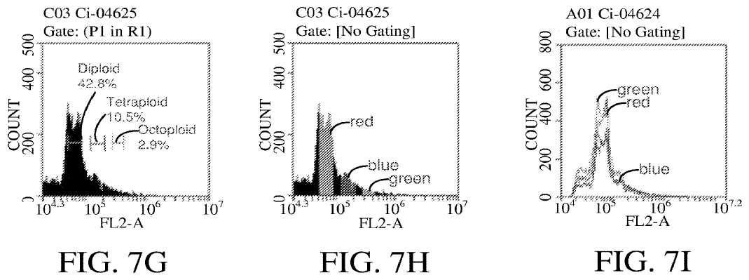

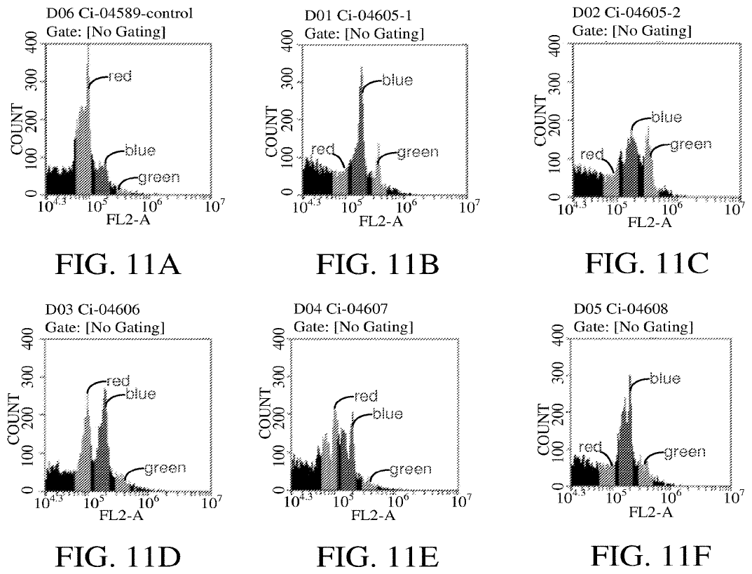

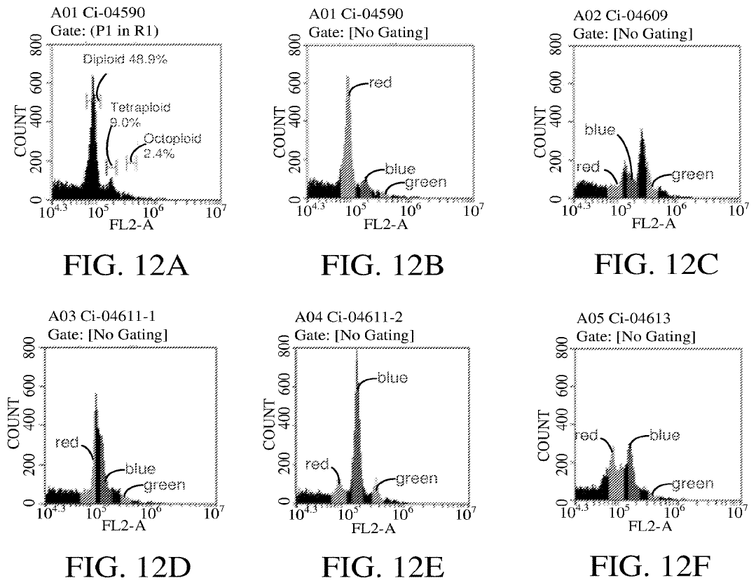

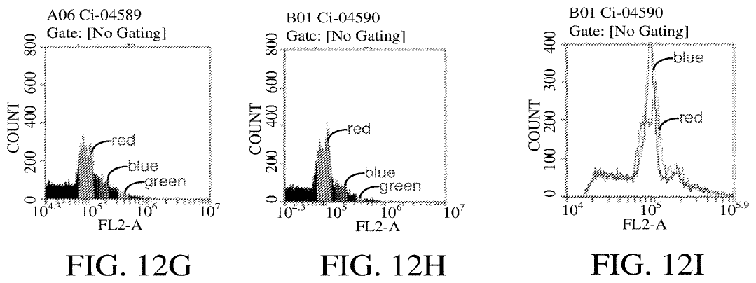

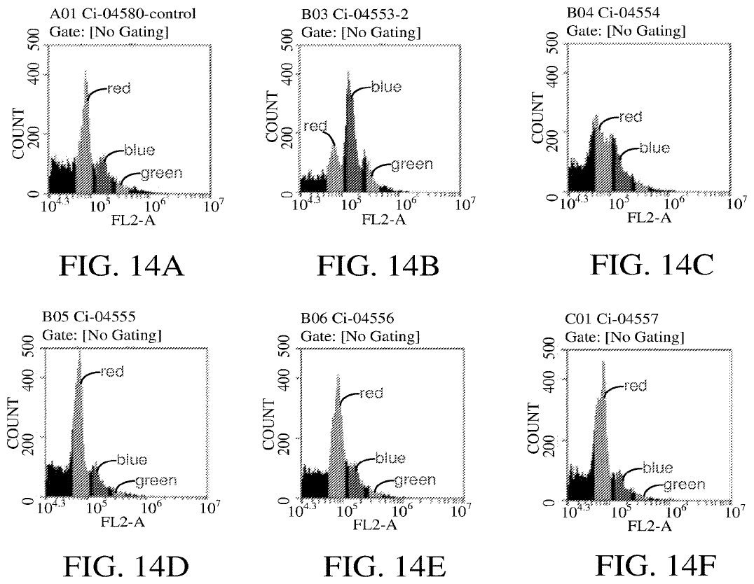

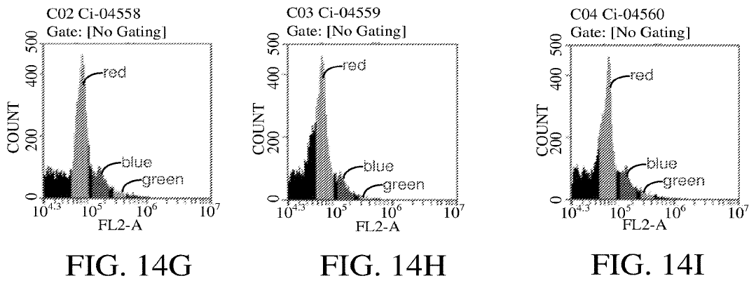

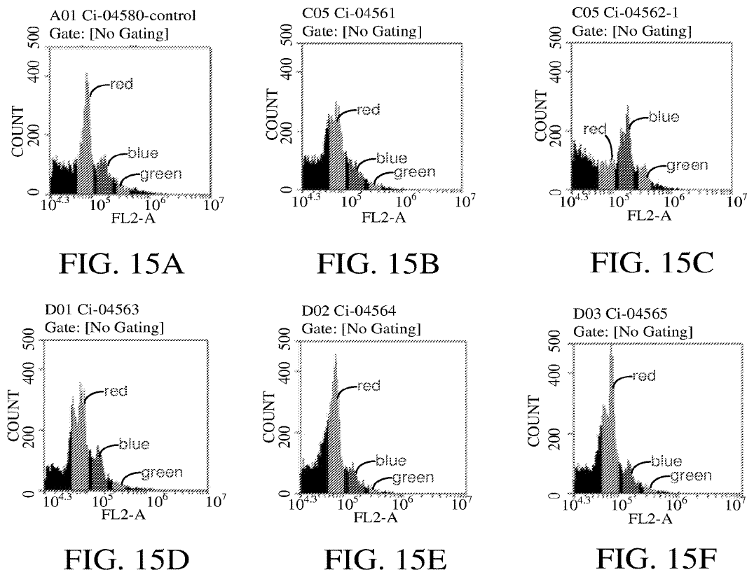

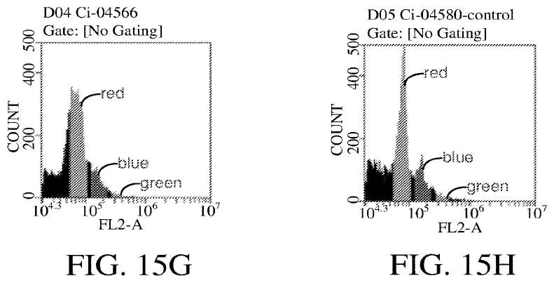

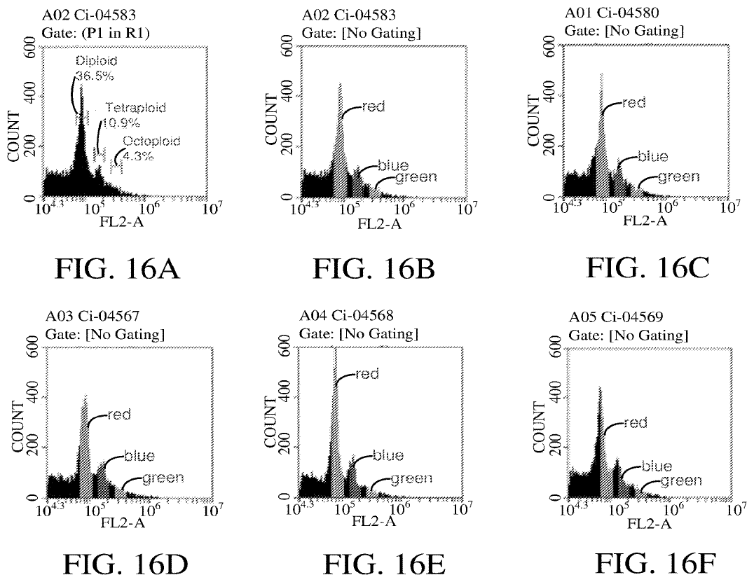

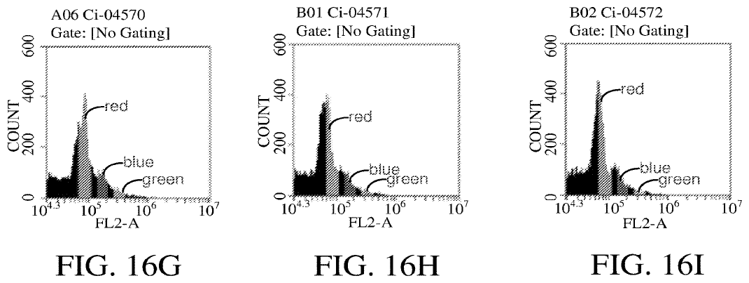

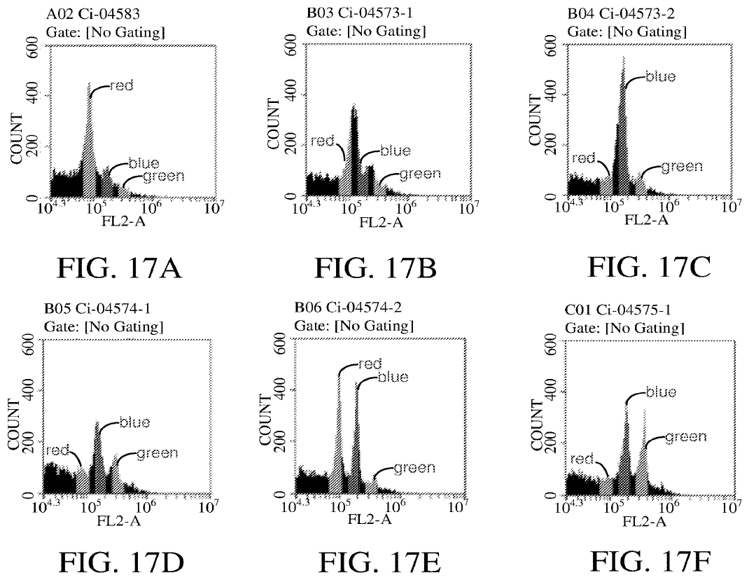

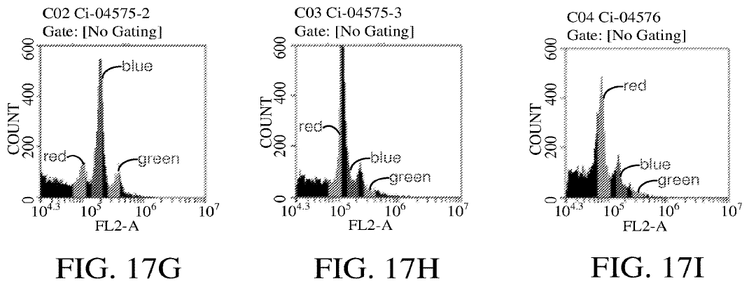

An experiment was conducted that involved treating Cannabis plants with different chemical solutions. The different treatments are shown below in Table 1. Each treatment group included a subgroup of Cannabis plants which were treated with a solution comprising a chemical agent, and a subgroup of Cannabis plants which were treated with a solution (e.g., water) without a chemical agent and used as a control. The treatment groups include Group 7A, 7B and 8. Group 7A and 7B included a subgroup of Cannabis plant clones that were treated with the solution of 0.25% w/v colchicine, 1% w/v Tween 20, and 2% w/v DMSO, and a subgroup of control Cannabis plant clones. Group 8 included a subgroup of Cannabis plant clones that were treated with a solution of 0.25% w/v colchicine and 1% w/v Tween 20 (e.g., without DMSO) and a subgroup of control Cannabis plant clones. As shown by Table 1, and further by FIGS. 5A-17I (and corresponding Appendices B-N of the underlying provisional application) the subgroups of Cannabis plants treated with the solution of 0.25% w/v colchicine, 1% w/v Tween 20, and 2% w/v DMSO, in Groups 7A and 7B, exhibited greater levels of doubling and tripling (and higher) chromosome events than the subgroups of control Cannabis plants in Groups 7A, 7B and 8 and the subgroup of cannabis plants treated with the solution of 0.25% w/v colchicine and 1% w/v Tween in Group 8. As the control Cannabis plants in Groups 7A, 7B and 8 were known to be diploid, the resulting range or peak in which the majority of nuclei fall for the control groups was used to define the (2n) diploid peaks for the treated plants. Relative to the control peak, ranges of peaks that fall in the 2n, 4n, and 8n range are colored in red, blue, and green, respectively and further described below.

TABLE 1

| | Mother | | | | |
|---|---|---|---|---|---|
| Sample | Line | Group | Treatment | Class. | FIG |
| Ci-04614-1 | PL 1 | 7A | 0.25% Colchicine + | Treated | 5B |
| | | | 1% Tween + 2% DMSO | | |
| Ci-04614-2 | PL 1 | 7A | 0.25% Colchicine + | Treated | 5C |
| | | | 1% Tween + DMSO | | |
| Ci-04616-1 | PL 1 | 7A | 0.25% Colchicine + | Treated | 5D |
| | | | 1% Tween + 2% DMSO | | |
| Ci-04616-2 | PL 1 | 7A | 0.25% Colchicine + | Treated | 5E |
| | | | 1% Tween + 2% DMSO | | |
| Ci-04617 | PL 1 | 7A | 0.25% Colchicine + | Treated | 5F |
| | | | 1% Tween + 2% DMSO | | |
| Ci-04618-1 | PL 1 | 7A | 0.25% Colchicine + | Treated | 5G |
| | | | 1% Tween + 2% DMSO | | |
| Ci-04618-2 | PL 1 | 7A | 0.25% Colchicine + | Treated | 5H |
| | | | 1% Tween + 2% DMSO | | |
| Ci-04620-1 | PL 1 | 7A | 0.25% Colchicine + | Treated | 5I |
| | | | 1% Tween + 2% DMSO | | |
| Ci-04620-2 | PL 1 | 7A | 0.25% Colchicine + | Treated | 6B |
| | | | 1% Tween + 2% DMSO | | |
| Ci-04622-1 | PL 1 | 7A | 0.25% Colchicine + | Treated | 6C |
| | | | 1% Tween + 2% DMSO | | |
| Ci-04622-2 | PL 1 | 7A | 0.25% Colchicine + | Treated | 6D |
| | | | 1% Tween + 2% DMSO | | |
| Ci-04624 | PL 1 | 7A | Control | Control | 6E, 7A, 7B, |
| | | | | | 7E, 7F, 7I |
| Ci-04625 | PL 1 | 7A | Control | Control | 5A, 6A, 6F, |
| | | | | | 7C, 7D, 7G, 7H |
| Ci-04589 | PL 2 | 7B | Control | Control | 8A, 8B, 8E, 8F, |
| | | | | | 8G, 8H, 8I, 9A, |
| | | | | | 10A, 11A, 12G |
| Ci-04590 | PL 2 | 7B | Control | Control | 8C, 8D, 12A, |
| | | | | | 12B, 12H, 12I |
| Ci-04591 | PL 2 | 7B | 0.25% Colchicine + | Treated | 9B |
| | | | 1% Tween + 2% DMSO | | |
| Ci-04592 | PL 3 | 7B | 0.25% Colchicine + | Treated | 9C |
| | | | 1% Tween + 2% DMSO | | |
| Ci-04593-1 | PL 3 | 7B | 0.25% Colchicine + | Treated | 9D |
| | | | 1% Tween + 2% DMSO | | |
| Ci-04593-2 | PL 3 | 7B | 0.25% Colchicine + | Treated | 9E |
| | | | 1% Tween + 2% DMSO | | |
| Ci-04596-1 | PL 2 | 7B | 0.25% Colchicine + | Treated | 9F |
| | | | 1% Tween + 2% DMSO | | |
| Ci-04596-2 | PL 2 | 7B | 0.25% Colchicine + | Treated | 9G |
| | | | 1% Tween + 2% DMSO | | |
| Ci-04597 | PL 2 | 7B | 0.25% Colchicine + | Treated | 9H |
| | | | 1% Tween + 2% DMSO | | |
| Ci-04598 | PL 2 | 7B | 0.25% Colchicine + | Treated | 91I |
| | | | 1% Tween + 2% DMSO | | |
| Ci-04599-1 | PL 2 | 7B | 0.25% Colchicine + | Treated | 10B |
| | | | 1% Tween + 2% DMSO | | |
| Ci-04600-1 | PL 2 | 7B | 0.25% Colchicine + | Treated | 10C |
| | | | 1% Tween + 2% DMSO | | |
| Ci-04600-2 | PL 2 | 7B | 0.25% Colchicine + | Treated | 10D |
| | | | 1% Tween + 2% DMSO | | |
| Ci-04602-1 | PL 2 | 7B | 0.25% Colchicine + | Treated | 10E |
| | | | 1% Tween + 2% DMSO | | |
| Ci-04602-2 | PL 2 | 7B | 0.25% Colchicine + | Treated | 10F |
| | | | 1% Tween + 2% DMSO | | |
| Ci-04604-1 | PL 3 | 7B | 0.25% Colchicine + | Treated | 10G |
| | | | 1% Tween + 2% DMSO | | |
| Ci-04604-2 | PL 3 | 7B | 0.25% Colchicine + | Treated | 10H |
| | | | 1% Tween + 2% DMSO | | |
| Ci-04604-3 | PL 3 | 7B | 0.25% Colchicine + | Treated | 10I |
| | | | 1% Tween + 2% DMSO | | |
| Ci-04605-1 | PL 3 | 7B | 0.25% Colchicine + | Treated | 11B |
| | | | 1% Tween + 2% DMSO | | |
| Ci-04605-2 | PL 3 | 7B | 0.25% Colchicine + | Treated | 11C |
| | | | 1% Tween + 2% DMSO | | |
| Ci-04606 | PL 2 | 7B | 0.25% Colchicine + | Treated | 11D |
| | | | 1% Tween + 2% DMSO | | |
| Ci-04607 | PL 2 | 7B | 0.25% Colchicine + | Treated | 11E |
| | | | 1% Tween + 2% DMSO | | |
| Ci-04608 | PL 2 | 7B | 0.25% Colchicine + | Treated | 11F |
| | | | 1% Tween + 2% DMSO | | |
| Ci-04609 | PL 2 | 7B | 0.25% Colchicine + | Treated | 12C |
| | | | 1% Tween + 2% DMSO | | |
| Ci-04611-1 | PL 2 | 7B | 0.25% Colchicine + | Treated | 12D |
| | | | 1% Tween + 2% DMSO | | |
| Ci-04611-2 | PL 2 | 7B | 0.25% Colchicine + | Treated | 12E |
| | | | 1% Tween + 2% DMSO | | |
| Ci-04613 | PL 3 | 7B | 0.25% Colchicine + | Treated | 12F |
| | | | 1% Tween + 2% DMSO | | |
| Ci-04547-1 | PL 4 | 8 | 0.25% Colchicine + | Treated | 13D |
| | | | 1% Tween | | |
| Ci-04547-2 | PL 4 | 8 | 0.25% Colchicine + | Treated | 13E |
| | | | 1% Tween | | |
| Ci-04548 | PL 4 | 8 | 0.25% Colchicine + | Treated | 13F |
| | | | 1% Tween | | |
| Ci-04550 | PL 4 | 8 | 0.25% Colchicine + | Treated | 13G |
| | | | 1% Tween | | |
| Ci-04552 | PL 4 | 8 | 0.25% Colchicine + | Treated | 13H |
| | | | 1% Tween | | |
| Ci-04553-1 | PL 4 | 8 | 0.25% Colchicine + | Treated | 13I |
| | | | 1% Tween | | |
| Ci-04553-2 | PL 4 | 8 | 0.25% Colchicine + | Treated | 14B |
| | | | 1% Tween | | |
| Ci-04554 | PL 5 | 8 | 0.25% Colchicine + | Treated | 14C |
| | | | % Tween | | |
| Ci-04555 | PL 5 | 8 | 0.25% Colchicine + | Treated | 14D |
| | | | 1% Tween | | |
| Ci-04556 | PL 4 | 8 | 0.25% Colchicine + | Treated | 14E |
| | | | 1% Tween | | |
| Ci-04557 | PL 4 | 8 | 0.25% Colchicine + | Treated | 14F |
| | | | 1% Tween | | |
| Ci-04558 | PL 4 | 8 | 0.25% Colchicine + | Treated | 14G |
| | | | 1% Tween | | |
| Ci-04559 | PL 5 | 8 | 0.25% Colchicine + | Treated | 14H |
| | | | 1% Tween | | |
| Ci-04560 | PL 5 | 8 | 0.25% Colchicine + | Treated | 14I |
| | | | 1% Tween | | |
| Ci-04561 | PL 5 | 8 | 0.25% Colchicine + | Treated | 15B |
| | | | 1% Tween | | |
| Ci-04562-1 | PL 5 | 8 | 0.25% Colchicine + | Treated | 15C |
| | | | 1% Tween | | |
| Ci-04563 | PL 5 | 8 | 0.25% Colchicine + | Treated | 15D |
| | | | 1% Tween | | |
| Ci-04564 | PL 5 | 8 | 0.25% Colchicine + | Treated | 15E |
| | | | 1% Tween | | |
| Ci-04565 | PL 5 | 8 | 0.25% Colchicine + | Treated | 15F |
| | | | 1% Tween | | |
| Ci-04566 | PL 5 | 8 | 0.25% Colchicine + | Treated | 15G |
| | | | 1% Tween | | |
| Ci-04567 | PL 4 | 8 | 0.25% Colchicine + | Treated | 16D |
| | | | 1% Tween | | |
| Ci-04568 | PL 4 | 8 | 0.25% Colchicine + | Treated | 16E |
| | | | 1% Tween | | |
| Ci-04569 | PL 4 | 8 | 0.25% Colchicine + | Treated | 16F |
| | | | 1% Tween | | |
| Ci-04570 | PL 4 | 8 | 0.25% Colchicine + | Treated | 16G |
| | | | 1% Tween | | |
| Ci-04571 | PL 4 | 8 | 0.25% Colchicine + | Treated | 16H |
| | | | 1% Tween | | |
| Ci-04572 | PL 4 | 8 | 0.25% Colchicine + | Treated | 16I |
| | | | 1% Tween | | |
| Ci-04573-1 | PL 4 | 8 | 0.25% Colchicine + | Treated | 17B |
| | | | 1% Tween | | |
| Ci-04573-2 | PL 4 | 8 | 0.25% Colchicine + | Treated | 17C |
| | | | 1% Tween | | |
| Ci-04574-1 | PL 4 | 8 | 0.25% Colchicine + | Treated | 17D |
| | | | 1% Tween | | |
| Ci-04574-2 | PL 4 | 8 | 0.25% Colchicine + | Treated | 17E |
| | | | 1% Tween | | |
| Ci-04575-1 | PL 4 | 8 | 0.25% Colchicine + | Treated | 17F |
| | | | 1% Tween | | |
| Ci-04575-2 | PL 4 | 8 | 0.25% Colchicine + | Treated | 17G |
| | | | 1% Tween | | |
| Ci-04575-3 | PL 4 | 8 | 0.25% Colchicine + | Treated | 17H |
| | | | 1% Tween | | |
| Ci-04576 | PL 4 | 8 | 0.25% Colchicine + | Treated | 17I |
| | | | 1% Tween | | |
| Ci-04580 | PL 4 | 8 | Control | Control | 13A, 13B, 14A, |
| | | | | | 15A, 15H, 16C |
| Ci-04583 | PL 4 | 8 | Control | Control | 13C, 16A, 16B, 17A |

plants with a Ci-##### that have the same # but different “-#” are shoots on the same plant

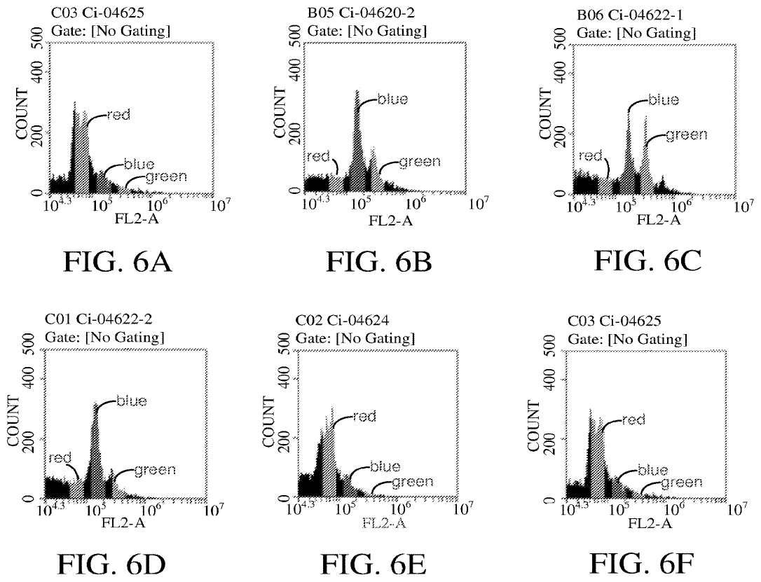

FIGS. 5A-7I are graphs illustrating resulting ploidy levels from Group 7A of the Table 1, consistent with the present disclosure. For example, FIGS. 5B-5I and FIGS. 6B-6F include graphs illustrating the resulting mixture of ploidy levels from the subgroup of Cannabis plants treated with the solution of 0.25% w/v colchicine, 1% w/v Tween 20, and 2% w/v DMSO in Group 7A as compared to the control Cannabis plants as illustrated by FIGS. 5A and 6A. FIGS. 5A, 6A, 7A-7I include graphs illustrating the resulting mixture of ploidy levels from the control Cannabis plants in Group 7A.

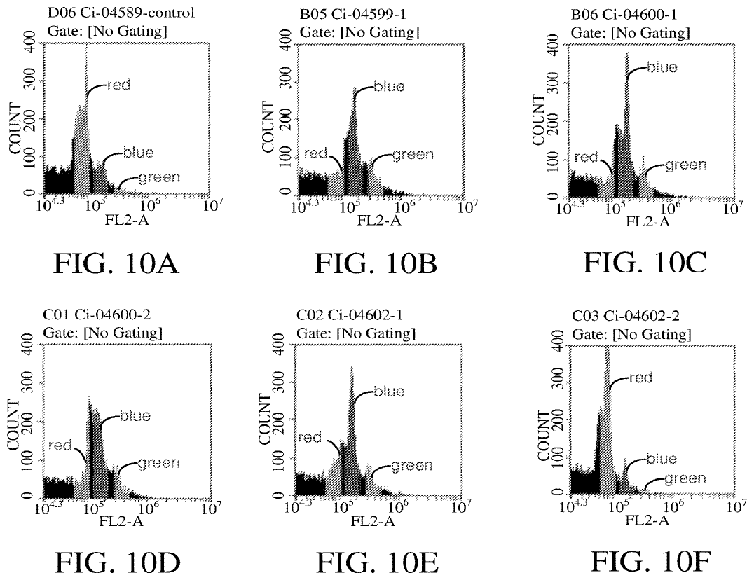

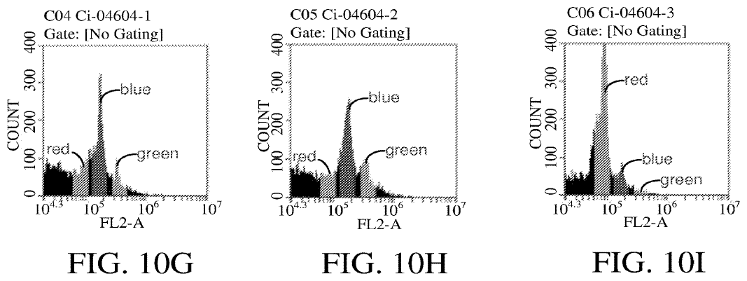

In each of the graphs illustrated by FIGS. 5A-17I, as well as FIGS. 18A-18C, FIGS. 19A-19T and FIGS. 10A-10E, the red peaks represent resulting peaks that are consistent with the control (2n) diploid plant parts. Relative to the control peaks, additional peaks are determined, such as the blue peaks that represent resulting (4n) tetraploid plant parts, the green peaks that represent resulting (8n) octoploid plant parts, and the black peaks between the respective red, blue, and green peaks that represent plant parts with values between (e.g., 3n, 5n, 6n, and 7n). For example, a black peak between a red peak (e.g., diploid) and a blue peak (e.g., tetraploid) represents resulting (3n) triploid plants. A black peak between a blue peak and a green peak (e.g., octoploid) represents resulting 5n, 6n, and/or 7n plants. In some experimental embodiments, one or more of the resulting plants (which are represented in the peaks) comprise aneuploid plants, such as resulting (3n) triploid plants having incomplete chromosome sets or more copies of one or more chromosomes of the chromosome sets, e.g., 29 chromosomes, 31 chromosomes, etc. In the various graphs, the Y-axis is the count of nuclei measured by the flow cytometer, and the X-axis is the strength of the fluorescence signature.

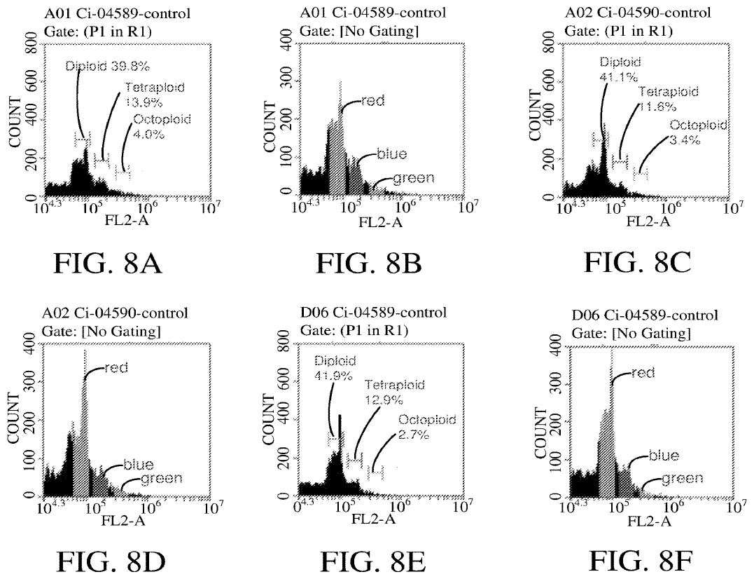

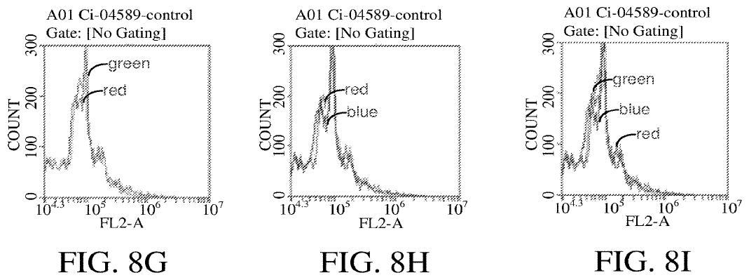

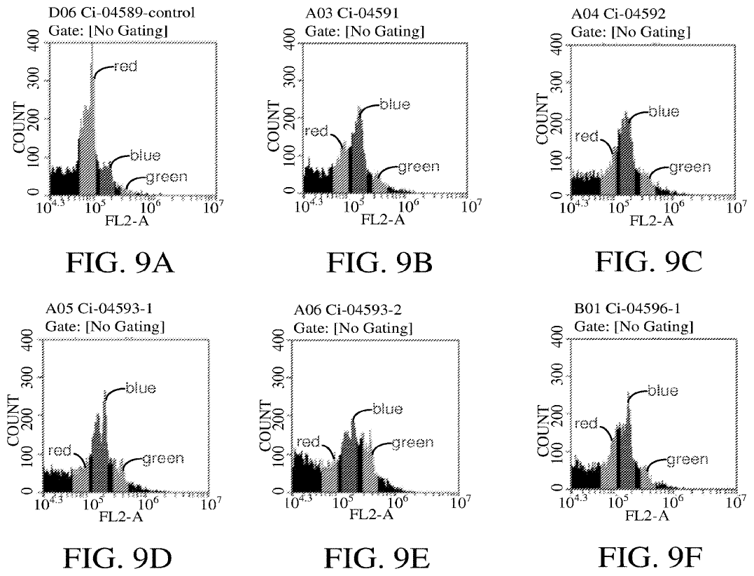

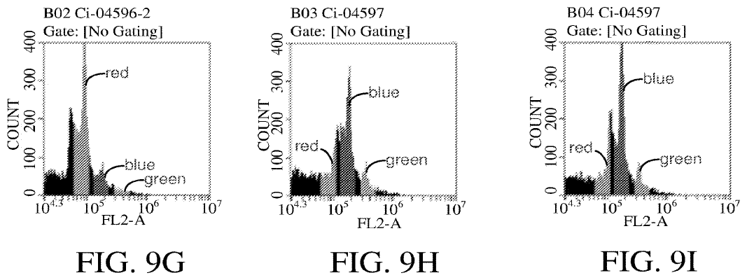

FIGS. 8A-12I are graphs illustrating resulting ploidy levels from Group 7B of the Table 1, consistent with the present disclosure. For example, FIGS. 9B-9I, FIGS. 10B-10I, FIGS. 11B-11F, and FIGS. 12C-12F include graphs illustrating resulting mixture of ploidy levels from the subgroup of Cannabis plants treated with the solution of 0.25% w/v colchicine, 1% w/v Tween 20, and 2% w/v DMSO in Group 7B as compared to the control Cannabis plants as illustrated by FIGS. 9A, 10A, 11A, 12A-12B, and 12G-12I. FIGS. 8A-8I (as well as FIGS. 9A, 10A, 11A, 12A-12B, and 12G-12I) include graphs illustrating the resulting mixture of ploidy levels from the control Cannabis plants in Group 7B.

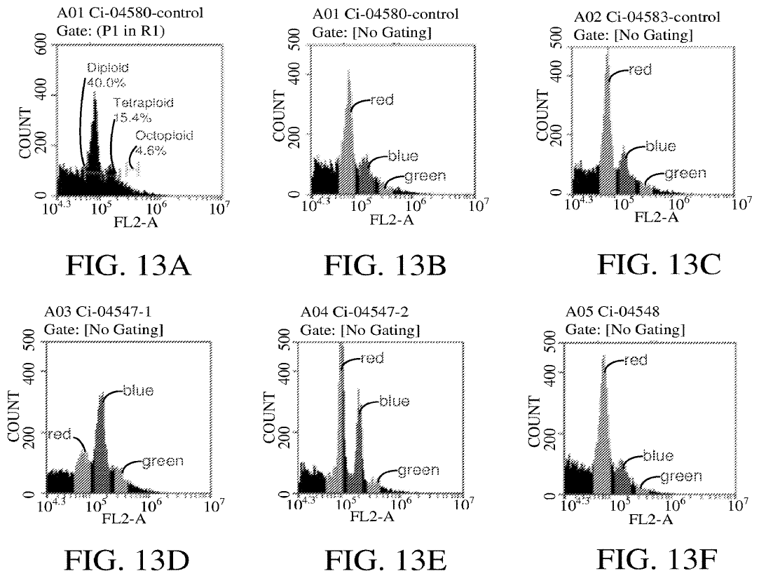

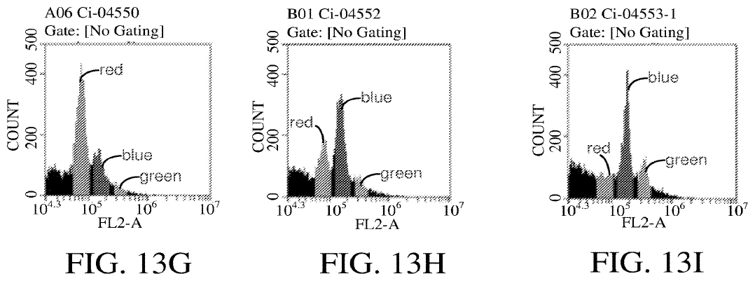

FIGS. 13A-17I are graphs illustrating resulting ploidy levels from Group 8 of the Table 1, consistent with the present disclosure. For example, FIGS. 13A-13I, FIG. 14A-14I, FIGS. 15A-15H, FIGS. 16A-16I, and FIGS. 17A-17I include graphs illustrating the resulting mixture of ploidy levels from the subgroup of Cannabis plants treated with the solution of 0.25% w/v colchicine and 1% w/v Tween 20 in Group 8, as well as the resulting mixture of ploidy levels from the control Cannabis plants in Group 8 (e.g., FIGS. 13A-13C, 14A, 15A, 16A-16C, 17A).

Additional experiments were conducted to illustrate selection of 4n cannabis plants, or other 2m ploidy levels, after treating with a solution of colchicine, Tween 20, and DMSO.

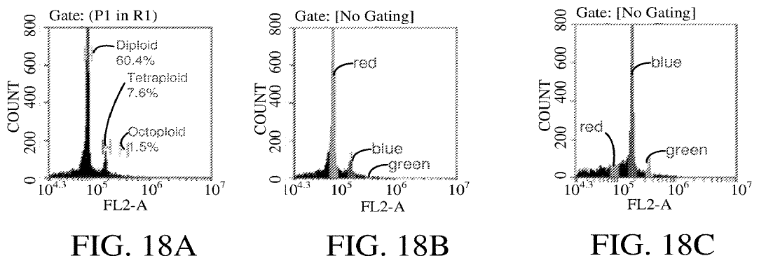

FIGS. 18A-20E are graphs illustrating resulting ploidy levels from Cannabis plants treated with a solution of colchicine, Tween 20, and DMSO as compared to resulting ploidy levels from control Cannabis plants, consistent with the present disclosure. FIGS. 18A-18B include graphs illustrating the resulting mixture of ploidy levels from the leaf tissue of a plant treated with water only (e.g., twice a day for three days) or no treatment, which was used as the control. As shown by FIG. 18A, for the control plants, the majority of the nuclei (60.4%) fall in a specific range, which for this known 2n control sample was used to define the (2n) diploid peak. Relative to the control peak, ranges of peaks that fall in the 2n, 4n, and 8n range are colored in red, blue, and green, as described above. FIG. 18B illustrates the same result as FIG. 18A without the annotations of percent. FIG. 18C includes a graph illustrating the resulting mixture of ploidy levels from the leaf tissue of a plant treated with the solution of 0.25% w/v colchicine, 1% w/v Tween 20, and 2% w/v DMSO, as described above (e.g., twice a day for three days). In the particular experiment, as shown by FIG. 18C, the resulting modified ploidy levels was 4n as the fluorescence distribution peak that falls primarily in the 4n range was twice the fluorescence strength of the 2n control.

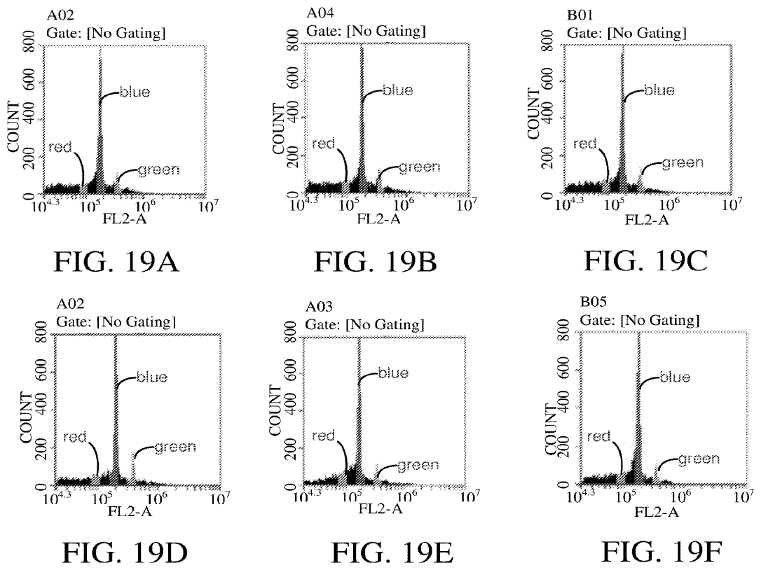

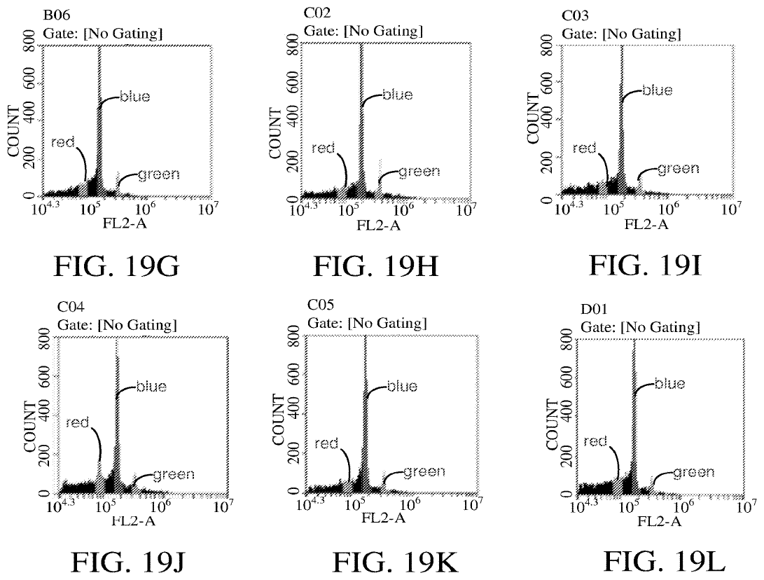

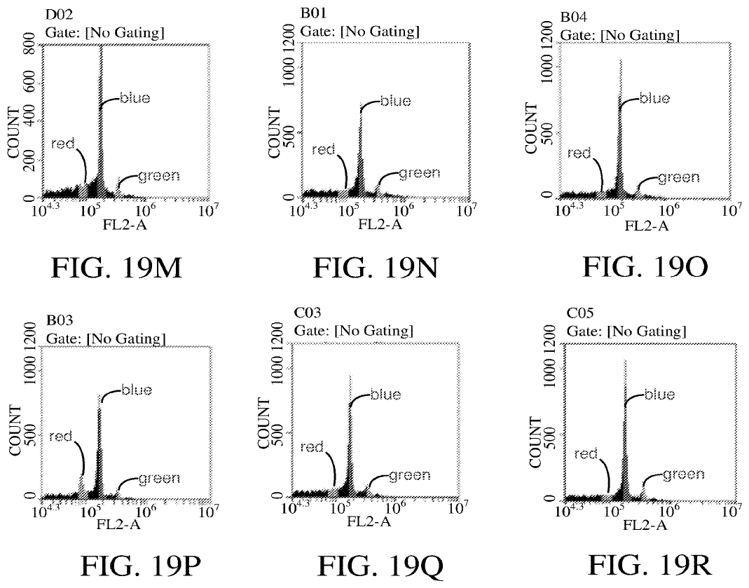

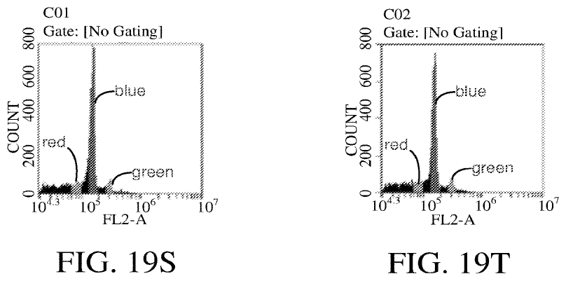

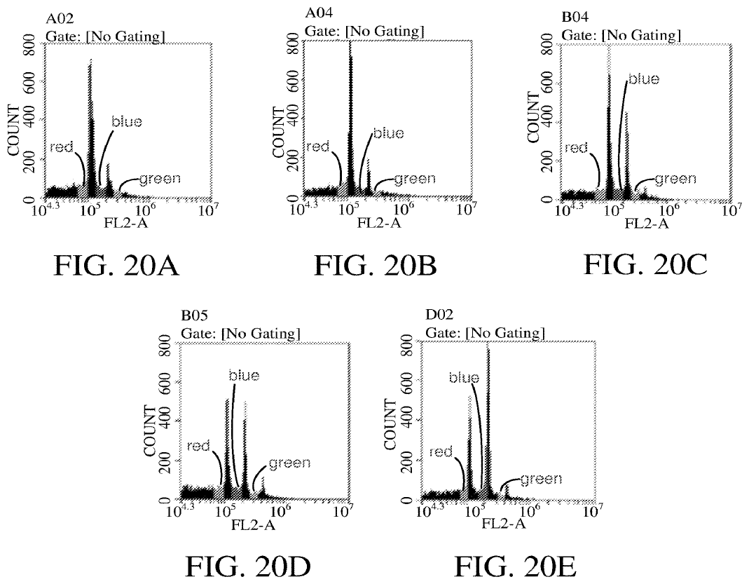

FIGS. 19A-19T and FIGS. 20A-20E are graphs illustrating resulting ploidy levels from leaf tissue of plants treated with the solution of colchicine, Tween 20, and DMSO. As shown, each of the graphs of FIGS. 19A-19T include a largest peak that is primarily in the 4n range, being twice the fluorescence strength of the 2n control, such as shown by FIGS. 18A-18B. Each of the graphs of FIGS. 20A-20E include one or more largest peaks that are between primarily between the 2n range and the 4n range and/or between the 4n range and the 8n range, indicating triploid and/or chimeric Cannabis plants.

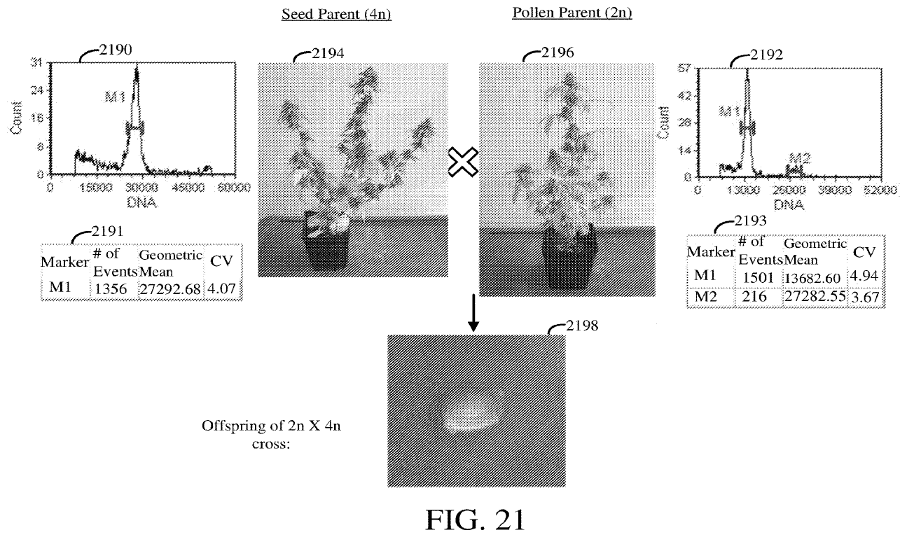

FIG. 21 illustrates an example of cross breeding Cannabis plants and a resulting offspring Cannabis plant part, consistent with the present disclosure. In various experimental embodiments, a 3n Cannabis plant was generated by crossing a 2n Cannabis plant with a 4n Cannabis plant. Each parent plant contributed half of its DNA to its gametes, with the 4n Cannabis plant generating 2n gametes and the 2n Cannabis plant generating 1n gametes. When the 2n gamete fuses with the 1n gamete, a 3n zygote is generated. The pollen donor can be either the 2n or the 4n plant. The following example experiment used the 2n plant as the pollen donor. However, embodiments are not so limited. Also in the example experimental embodiment, only genetically female plants are used, so the process of generating feminized pollen is used to make pollen from female plants.

As shown by the flow cytometry graph 2192 and the table 2193 of FIG. 21, the 2n Cannabis parent plant has a similar primary fluorescence peak position to the 2n control plants in Table 1 and as illustrated by at least the graph of FIG. 5A and the graph 2235 of FIG. 221 below (e.g., red peak), showing that the Cannabis plant is diploid. As shown by the flow cytometry graph 2190 and the table 2191 of FIG. 21, the 4n Cannabis parent plant has a primary fluorescence peak position at twice the primary fluorescence peak position of the 2n Cannabis plants (e.g., blue peak), showing that the Cannabis plant is tetraploid. In the experimental embodiment, the 4n Cannabis parent plant was crossed with the 2n cannabis parent plant, as illustrated by the image of the 4n Cannabis parent plant 2194 and the image of the 2n Cannabis parent plant 2196. The result of the 2n by 4n cross was an offspring Cannabis plant part, as shown by the image of the resulting seed 2198 (e.g., 3n seed).

To generate and collect feminized pollen, the following was performed. When the 2n Cannabis plants were at least six inches tall, the Cannabis plants were placed in 12 hour light-12 hour dark conditions to induce flowering. The foliar portions of the plants were thoroughly sprayed twice a week with a 5.6 millimolar (mM) concentration of Silver Thiosulfate (STS) just before the lights are shut off for the day. After about four to six weeks of the STS treatment, pollen sacs began to form on the plants. When the pollen sacs were open or just about to open, forceps were used to gently remove the pollen sacs from the plant and the pollen sacs were placed into a 5 ml tube. Optionally, to promote desiccation, as well adding friction to break open the pollen sacs, the tubes were prefilled with about 10% filled with dry rice. In some experiments, the tube was filled to about 70% volume with pollen sacs and rice. To encourage pollen dehiscence, the tube cap was firmly closed and the tube was shaken. In some embodiments, optionally (though not required if sufficient pollen is present), the pollen sacs can be removed from the pollen by filtering with a mesh sized to catch the pollen sacs and rice while allowing the pollen to fall through onto a collection paper. The pollen can then be transferred to a new tube. Some pollen will likely be lost in the filtering and transfer process.

To generate female flowers, the following was performed. When the 4n cannabis plants were at least six inches tall, the Cannabis plants were placed in 12 hour light-12 hour dark conditions to induce flowering. After about four weeks, the plants began to produce female flowers.

Embodiments are not limited to using the 2n Cannabis plant as the pollen donor. In some embodiments, the 4n Cannabis plant is used as the pollen donor.

In various experimental embodiments, the 2n Cannabis plants and the 4n cannabis plant are crossed using the produced pollen and female flowers. For example, the feminized pollen was generated and collected as described above. The produced female flowers were screened to select female flowers with long, turgid, white pistils. Using a cotton swab (e.g., a cotton end of a stick), pollen was gently picked up from the pollen tube and the cotton swab was gently touched to the pistils. When finished, some pollen grains were visible on the pistils. The pollination process was repeated for additional flowers on the Cannabis plant, preferably working systematically branch by branch. For each branch or stem section that was pollinated, a crossing tag was applied to the base of the stem or branch that indicated the assigned cross population number, the male and female parent, and the crossing date. To prevent additional accidental pollinations, the pollinated branch was covered using a glassine bag. An angle fold in the bag was used to create a tight seal at the base of the stem or branch and the folded section of the bag was stapled to hold the bag in place. The bag was marked with the crossing date. Five days after making the cross, the pollinated branch was removed the crossing bag. Before removing the bag, a small slit in the bag was created and a spray bottle was used to mist the inside of the crossing bag with water to kill any pollen inside. Alternatively, in order to increase seed set, bags can be removed one or two days after pollination and additional pollinations can be conducted (using the same pollen donor). The seed should mature about four to five weeks after pollination.

Various experimental embodiments can be conducted to evaluate the ploidy levels and phenotypes of the resulting offspring after the pollination. An example experiment, to test to evaluate certain phenotypes of 3n Cannabis plants can include five groups of plants. Groups 1-4 can include plants grown to generate female flowers, and these groups can vary by ploidy level and if the plant is to be pollinated or not pollinated (see Table 2 below). Group 5 plants can be grown to generate feminized pollen used to pollinate Groups 2 and 4. Hemp flowers (colas) from each of the Groups 1-4 can be sampled at roughly weekly intervals after the initiation of flowering. THC, CBD, and other cannabinoids can be assayed using HPLC.

TABLE 2

| Group | Ploidy | Pollinated/Not Pollinated | Compared to Group(s) |
|---|---|---|---|
| 1 | 3n | Not-Pollinated | 2, 3, & 4 |
| 2 | 3n | Pollinated | 1, 3, & 4 |
| 3 | 2n | Not-Pollinated | 1, 2, & 4 |
| 4 | 2n | Pollinated | 1, 2, & 3 |
| 5 | 2n | (Pollen producers) | N/A |

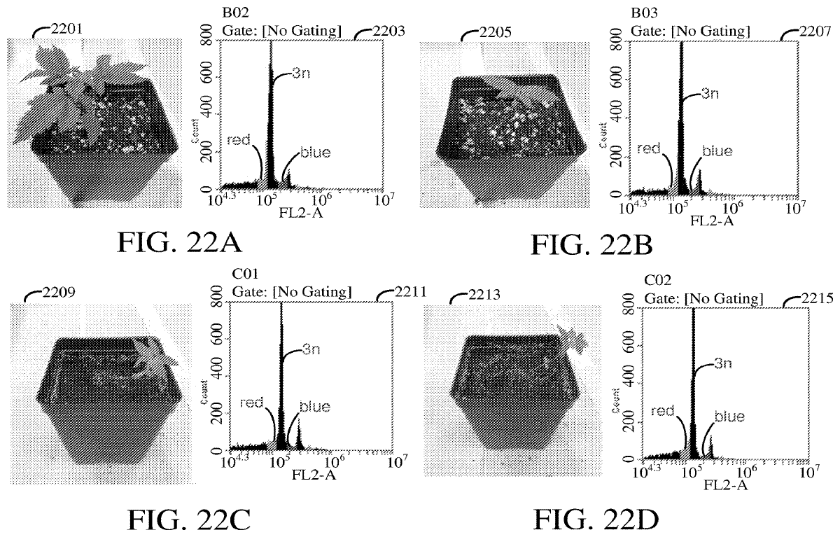

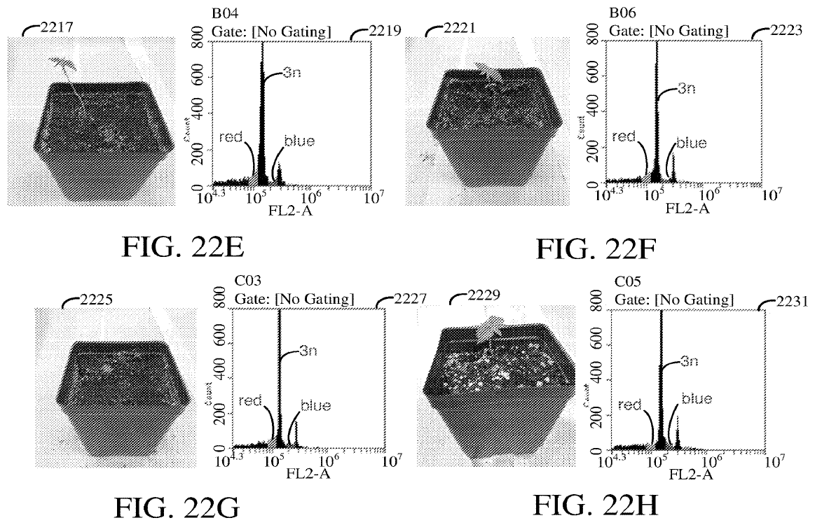

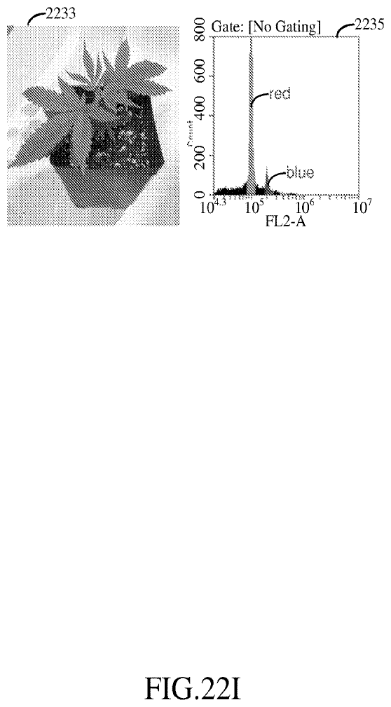

FIGS. 22A-22I illustrate example offspring from cross breeding Cannabis plants, consistent with the present disclosure. In various experimental embodiments, a 2n pollen donor Cannabis parent was crossed with a 4n ovule donor Cannabis parent (e.g., a female 4n Cannabis parent) to generate Cannabis offspring, such as described above by FIG. 21. The resulting Cannabis offspring, as illustrated by FIGS. 22A-22H were (3n) triploid offspring. FIGS. 22A-22H illustrate images of eight independent triploid Cannabis offspring 2201, 2205, 2209, 2213, 2217, 2221, 2225, 2229 generated from the 4n by 2n cross and resulting flow cytometry graphs 2203, 2207, 2211, 2215, 2219, 2223, 2227, 2231. The resulting cannabis offspring were triploid as illustrated by the primary peak between the red peak and blue peak in each of the flow cytometry graphs 2203, 2207, 2211, 2215, 2219, 2223, 2227, 2231. FIG. 221 illustrates an example flow cytometry graph 2235 of a control Cannabis plant and an image of the control Cannabis plant 2233 (e.g., a 2n Cannabis plant).

The above described experimental embodiments demonstrate chemical treatment of Cannabis plants/plant parts, which successfully resulted in different modified ploidy levels. The different modified ploidy levels included 3n, 4n, and 8n, among others as shown by the various graphs. The experimental embodiments additionally demonstrate successfully cross breeding a Cannabis plant exhibiting a modified ploidy level (from the chemical treatment) with a 2n Cannabis plant to generate offspring (e.g., seeds, plants), such as 3n cannabis offspring plant. Embodiments in accordance with the present disclosure are not limited to that demonstrated by the experimental embodiments and can include a variety of different variations of chemical treatment, and can include or not include cross breeding to generate a 2p+1 ploidy level Cannabis plant, plant part, or plant cell.

1. A method, comprising:
treating a Cannabis plant, plant part, or plant cell with a solution including a chemical agent configured to modify a ploidy level of the Cannabis plant, plant part, or plant cell; and
in response to the treatment, modifying the ploidy level of the Cannabis plant, plant part, or plant cell.

2. The method of claim 1, wherein treating the Cannabis plant, plant part, or plant cell with the solution includes applying the solution to at least a portion of the Cannabis plant, the plant part, or the plant cell, wherein the solution includes the chemical agent, a surfactant and, a polar aprotic solvent.

3. The method of claim 2, wherein the surfactant is a foaming agent, and treating the Cannabis plant, plant part, or plant cell with the solution includes applying the solution at least partially in a foam form to the Cannabis plant, plant part, or plant cell.

4. The method of claim 2, wherein the surfactant is Tween 20, and the polar aprotic solvent is Dimethylsulphoxide (DMSO).

5. The method of claim 1, wherein the chemical agent is selected from colchicine, acenaphtene, trifluralin, aminoprophosmethyl, pronamide, oryzalin, and nitrous oxide.

6. The method of claim 1, further including selecting the Cannabis plant, plant part, or plant cell from a plurality of Cannabis plants, plant parts, or plant cells treated with the solution by identifying the modified ploidy level of the Cannabis plant, plant part, or plant cell includes 2m or 2p+1 complete chromosome sets, incomplete chromosome sets, or chromosome sets with an additional copy of one or more chromosomes in the chromosome sets,
wherein when the modified ploidy level is 2m, m is at least 2.

7. The method of claim 6, wherein;
when the modified ploidy level is 2p+1, p is an integer from 1 to 4; and
when the modified ploidy level is 2m, m is an integer from 2 to 4.

8. The method of claim 1, wherein the modified ploidy level of the Cannabis plant, plant part, or plant cell includes a tetraploid level, and the method further includes cross breeding a diploid Cannabis plant and the Cannabis plant having the modified ploidy level.

9. The method of claim 8, further including identifying, from the cross breed of the diploid Cannabis plant and the Cannabis plant, at least one offspring Cannabis plant that is triploid and female.

10. A method, comprising;
treating a plurality of Cannabis plants, plant parts, or plant cells with a solution including a chemical agent configured to modify a ploidy level of the Cannabis plants, plant parts, or plant cells;
selecting at least one of the plurality of Cannabis plants, plant parts, or plant cells that exhibits the modified ploidy level, the modified ploidy level including 2m or 2p+1 complete chromosome sets, incomplete chromosome sets, or chromosome sets with an additional copy of one or more chromosomes in the chromosome sets; and
wherein when the modified ploidy level is 2m, m is at least 2.

11. The method of claim 10, wherein when the modified ploidy level is 2p+1, p is an integer from 1 to 4; and
when the modified ploidy level is 2m, m is an integer from 2 to 4.

12. The method of claim 10, wherein the modified ploidy level includes a diploid level or a triploid level.

13. The method of claim 10, further including cross breeding a diploid Cannabis plant and the Cannabis plant, or a modified Cannabis plant generated from the plant part or plant cell that exhibits the modified ploidy level, to generate an offspring Cannabis plant.

14. The method of claim 13, further including selecting the offspring Cannabis plant, the selected offspring Cannabis plant including a triploid Cannabis plant.

15. The method of claim 14, wherein the selected offspring Cannabis plant is female and sterile.

16. The method of claim 14, further including generating clones of the selected offspring Cannabis plant.

17. The method of claim 10, wherein the selected at least one of the plurality of cannabis plants, plant parts, or plant cells is a triploid Cannabis plant.

18. The method of claim 10, wherein treating the plurality of Cannabis plants, plant parts, or plant cells with the solution includes applying the solution at least partially in a foam form to the plurality of Cannabis plants, plant parts, or plant cells, wherein the solution includes the chemical agent, a surfactant and, a polar aprotic solvent.

19. The method of claim 18, wherein:
the surfactant is Tween 20;
the polar aprotic solvent is Dimethylsulphoxide (DMSO); and
the chemical agent is selected from colchicine, acenaphtene, trifluralin, aminoprophosmethyl, pronamide, oryzalin, and nitrous oxide.

20. A Cannabis plant, Cannabis plant part, or Cannabis plant cell having 2p+1 complete chromosome sets, incomplete chromosome sets, or chromosome sets with an additional copy of one or more chromosomes in the chromosome sets.
